(19) United States
(12) Patent Application Publication
(10) Pub. No.: US 2024/0285670 A1
(43) Pub. Date: Aug. 29, 2024

(54) THERAPEUTIC TREATMENT OF TUMORS BY BLOCKING THE HEME SYNTHESIS-EXPORT AXIS

(71) Applicant: Università degli Studi di Torino, Torino (IT)

(72) Inventors: Emanuela TOLOSANO, Torino (IT); Veronica FIORITO, Savigliano (CN) (IT); Sara PETRILLO, Torino (IT); Deborah CHIABRANDO, Saluzzo (CN) (IT); Anna Lucia ALLOCCO, Fossano (CN) (IT); Francesca BERTINO, Torino (IT)

(21) Appl. No.: 18/568,541

(22) PCT Filed: Jun. 7, 2022
(86) PCT No.: PCT/IB2022/055291

§ 371 Date: Dec. 8, 2023

(30) Foreign Application Priority Data
Jun. 11, 2021 (IT) 102021000015368

(51) Int. Cl. A61K 31/713; A61K 31/197; A61P 35/00; C12N 15/113

(52) U.S. Cl. CPC A61K 31/713; A61K 31/197; A61P 35/00; C12N 15/1137; C12N 15/1138

(57) ABSTRACT

A blocker of heme synthesis-export axis as active agent for use in the treatment of a tumor in a subject, wherein the blocker of heme synthesis-export axis upmodulates oxidative metabolism of tumor cells by upregulating the tricarboxylic acid (TCA) cycle and the oxidative phosphorylation (OXPHOS).

### FIELD OF THE INVENTION

The present description concerns a new strategy for the therapeutic treatment of tumors wherein the active agent is a blocker of heme synthesis-export axis.

### BACKGROUND ART

Heme is an iron-containing porphyrin of vital importance for cells due to its involvement in several biological processes. Heme can be acquired from dietary sources, but it is also synthetized directly by cells. Heme synthesis consists of eight enzymatic reactions starting in mitochondria with the condensation of succinyl-CoA with glycine to form 5-aminolevulinic acid (ALA). This reaction is catalyzed by 5-aminolevulinate synthase (ALAS), the rate-limiting enzyme of the heme synthetic pathway. Two genes encode the ALAS enzyme, ALAS1, which is ubiquitously expressed, and ALAS2, which is specific for the erythroid lineage. Together with endogenous biosynthesis, cellular heme homeostasis relies on the balanced and coordinated expression/activity of enzymes, transporters and accessory proteins involved in extracellular heme import, heme incorporation into hemoproteins, heme degradation, and heme export from the cytosol to the extracellular space. This latter activity is mediated by the cell surface Feline Leukemia Virus subgroup C Receptor 1a (FLVCR1a), one of the two proteins encoded by the FLVCR1 gene.

Heme has profound and complex implications in processes related to cell energy production. Indeed, heme is involved in oxygen transport and plays pivotal functions in mitochondria, serving as a cofactor for most of the respiratory chain complexes. Moreover, heme biosynthesis is considered a cataplerotic pathway for the tricarboxylic acid (TCA) cycle, as the process consumes succinyl-CoA, an intermediate of the TCA cycle.

The control of cell energy metabolism and of nutrient expenditure to sustain energy production is particularly important in cells under conditions of high-energy demand, such as during proliferation. Therefore, it is not surprising that alterations of heme metabolism are frequently observed in cancer. It is commonly assumed that most tumors rely on high heme synthesis by ALAS1. The expression and/or activity of the heme biosynthetic enzymes ALAS1, porphobilinogen deaminase (PBGD) and uroporphyrinogen III decarboxylase (UROD) were frequently found up-regulated in cancer. Consistently, repression of heme biosynthesis by the delta-aminolevulinic acid dehydratase (ALAD) inhibitor succinylacetone was shown to reduce tumor cell survival and proliferation. Moreover, in nineties, it was discovered that tumors, upon ALA administration, are able to accumulate remarkably higher amount of protoporphyrin IX (PpIX) as compared to normal tissues, and this property was demonstrated to be exploitable for tumor fluorescence-guided surgery (FGS) and to kill cancer cells by photodynamic therapy (PDT) (Malik and Lugaci, 1987, Kennedy et al., 1990, Peng et al., 1992). Likewise, increased expression of the heme exporter FLVCR1a has been recently reported (Russo et al., 2019, Shen et al., 2018, Peng et al., 2018).

However, the precise mechanisms underpinning enhanced heme synthesis and enhanced heme export in tumors remain substantially unexplored. Experimental evidence suggests that heme synthesis by ALAS1 and heme export by FLVCR1a are two highly coordinated processes (Vinchi et al., 2014). Yet, the two processes have never been studied reciprocally in the context of cancer, and their impact on the global regulation of cell energy metabolism has not been explored.

### SUMMARY OF THE INVENTION

The object of this disclosure is to provide a new strategy for the treatment of tumors by blocking the heme synthesis-export axis.

According to the invention, the above object is achieved thanks to the subject matter recalled specifically in the ensuing claims, which are understood as forming an integral part of this disclosure.

The present invention concerns a blocker of heme synthesis-export axis as active agent for use in the treatment of a tumor in a subject, wherein the blocker of heme synthesis-export axis upmodulates oxidative metabolism of tumor cells by upregulating the tricarboxylic acid (TCA) cycle and the oxidative phosphorylation (OXPHOS).

In one embodiment, the present invention concerns a pharmaceutical composition comprising as active agent a blocker of heme synthesis-export axis for use in the treatment of a tumor in a subject, wherein the blocker of heme synthesis-export axis upmodulates oxidative metabolism of tumor cells by upregulating the tricarboxylic acid (TCA) cycle and the oxidative phosphorylation (OXPHOS).

In the present disclosure it is documented that ALAS1-mediated heme synthesis and FLVCR1a-mediated heme export are intertwined processes and that the heme synthesis-export axis is crucial for controlling the TCA cycle and oxidative phosphorylation (OXPHOS). Moreover, evidence is provided that the forced impairment of the heme synthesis-export system, by both an FLVCR1a-specific shRNA or, paradoxically, by the treatment with the heme precursor 5-ALA, result in reduced tumor cells proliferation and survival.

### BRIEF DESCRIPTION OF THE DRAWINGS

The invention will now be described in detail, purely by way of an illustrative and non-limiting example and, with reference to the accompanying drawings, wherein:

FIG. 1. FLVCR1a silencing by a specific shRNA.

Cells in which the expression of FLVCR1a was down-regulated using a specific shRNA are compared to cells expressing a scramble shRNA. (A, C, E) qRT-PCR analysis of the expression of FLVCR1a in Caco2 (A), SKCO1 (C) and C80 (E) cells. Transcript abundance, normalized to beta-actin mRNA expression, is expressed as a fold increase over a calibrator sample. Data represent mean±SEM, n=5 for SKCO1 cells, n=6 for Caco2 and C80 cells. For statistical analyses, an unpaired Student's t-test was used; **p<0.01, ***p<0.001. (B, D, F) Western blot analysis of FLVCR1a expression in Caco2 (B), SKCO1 (D) and C80 (F) cells. Vinculin expression is shown as a loading control. Band intensities were measured by densitometry and normalized to vinculin expression. Densitometry data represent mean±SEM, n=2. For statistical analyses, an unpaired Student's t-test was used; *=p<0.05, **=p<0.01. Data are representative of three independent experiments.

FIG. 2. The FLVCR1a-mediated heme export sustains heme synthesis.

(A) Heme content in Caco2 cells treated with 5 mM 5-ALA (black bars and white bars) or with 5 mM 5-ALA+0.5 mM SA (light grey outlined bars) for the indicated time points. Cells in which the expression of FLVCR1a was down-regulated using a specific shRNA are compared to cells expressing a scramble shRNA. Values are expressed as relative fluorescence intensity. Data represent mean±SEM, n=2. For statistical analyses, a two-way analysis of variance was used, followed by the Bonferroni correction for multiple group comparisons; **=p<0.01, ***=p<0.001. 5-ALA, 5-aminolevulinic acid; SA, succinylacetone. (B) Intracellular 5-ALA levels. Caco2 cells in which the expression of FLVCR1a was down-regulated using a specific shRNA are compared to cells expressing a scramble shRNA. Values are expressed as peak area/μg proteins. Data represent mean±SEM of n=4 wells pooled from two independent experiments. For statistical analyses, an unpaired Student's t-test was used; **=p<0.01. 5-ALA, 5-aminolevulinic acid. (C, D) Accumulation of ¹³C₂-5-ALA in cells incubated with U-¹³C-glucose (C) or of ¹³C₃-5-ALA in cells incubated with U-¹³C-glutamine (D) for the indicated time. Caco2 cells in which the expression of FLVCR1a was down-regulated using a specific shRNA are compared to cells expressing a scramble shRNA. Values are expressed as peak area/μg proteins. Data represent mean±SEM of n=4 wells pooled from two independent experiments. For statistical analyses, a two-way analysis of variance was used, followed by the Bonferroni correction for multiple group comparisons; ***p<0.001. 5-ALA, 5-aminolevulinic acid. (E, F) qRT-PCR analysis of the expression of ALAS1 in SKCO1 (E) and C80 (F) cells. Cells in which the expression of FLVCR1a was down-regulated using a specific shRNA are compared to cells expressing a scramble shRNA. Transcript abundance, normalized to beta-actin mRNA expression, is expressed as a fold increase over a calibrator sample. Data represent mean±SEM, n=5 for C80 cells and n=6 for SKCO1 cells; For statistical analyses, an unpaired Student's t-test was used; *=p<0.05, **=p<0.01. (G) Western blot analysis of ALAS1 expression in Caco2 cells. Cells overexpressing FLVCR1a (indicated as FLVCR1a+) are compared to cells stably transduced with an empty vector. Vinculin expression is shown as a loading control. Band intensities were measured by densitometry and normalized to vinculin expression. Densitometry data represent mean±SEM, n=2. For statistical analyses, an unpaired Student's t-test was used; *=p<0.05. Data are representative of three independent experiments.

FIG. 3. The heme synthesis-export system controls the OXPHOS rate.

(A, C, E) Activities of the mitochondrial electron transport chain complexes I-IV. FLVCR1a-silenced Caco2 (A), SKCO1 (C) or C80 cells (E) are compared to cells expressing a scramble shRNA. Results were expressed as nmol NAD⁺/min/mg mitochondrial protein for complex I, nmol reduced cytochrome c/min/mg mitochondrial protein for complexes II-III, nmol oxidized cytochrome c/min/mg mitochondrial protein for complex IV. Data represent mean±SEM, n=3. For statistical analyses, an unpaired Student's t-test was used; *=p<0.05, **=p<0.01. (B, D, F) Mitochondrial ATP levels measured by a bioluminescent assay kit. FLVCR1a-silenced Caco2 (B), SKCO1 (D) or C80 cells (F) are compared to cells expressing a scramble shRNA. Results are expressed as nmol/mg mitochondrial proteins. Data represent mean±SEM, n=3. For statistical analyses, an unpaired Student's t-test was used; **=p<0.01. (G) Activities of the mitochondrial electron transport chain complexes I-IV in Caco2 cells overexpressing FLVCR1a (indicated as FLVCR1a+) compared to cells stably transduced with an empty vector. Results were expressed as nmol NAD⁺/min/mg mitochondrial protein for complex I, nmol reduced cytochrome c/min/mg mitochondrial protein for complexes II-III, nmol oxidized cytochrome c/min/mg mitochondrial protein for complex IV. Data represent mean±SEM, n=3. For statistical analyses, an unpaired Student's t-test was used; *=p<0.05, **=p<0.01. (H) Mitochondrial ATP levels measured by a bioluminescent assay kit in Caco2 cells overexpressing FLVCR1a (indicated as FLVCR1a+) compared to cells stably transduced with an empty vector. Results are expressed as nmol/mg mitochondrial proteins. Data represent mean±SEM, n=3. For statistical analyses, an unpaired Student's t-test was used; *=p<0.05.

FIG. 4. The heme synthesis/export system controls the TCA cycle flux.

(A) Caco2 cells in which the expression of FLVCR1a was down-regulated using a specific shRNA are compared to cells expressing a scramble shRNA. TCA cycle flux is reported in the centre of the figure and expressed as pmol CO₂/h/mg protein. Data represent mean±SEM, n=3. For statistical analyses, an unpaired Student's t-test was used. **=p<0.01. Moreover, TCA cycle enzymes activities are reported in boxes and expressed as mU/mg mitochondrial proteins. Data represent mean±SEM, n=3. For statistical analyses, an unpaired Student's t-test was used; *=p<0.05, ***=p<0.001. (B, C) TCA cycle flux in SKCO1 (B) and C80 (C) cells in which the expression of FLVCR1a was down-regulated using a specific shRNA, compared to cells expressing a scramble shRNA. Results are expressed as pmol CO₂/h/mg protein. Data represent mean±SEM, n=3. For statistical analyses, an unpaired Student's t-test was used; *=p<0.05, **=p<0.01.

FIG. 5. The heme synthesis/export system controls the TCA cycle flux through ALAS1 regulation.

(A) TCA cycle flux in Caco2 cells expressing a scramble shRNA untreated or treated with 25 μM hemin for 2 hours. Moreover, the TCA cycle flux of untreated FLVCR1a-silenced Caco2 cells is reported for comparison. Results are expressed as pmol CO₂/h/mg protein. Data represent mean±SEM, n=3. For statistical analyses, an unpaired Student's t-test was used to compare the two matched groups; *=p<0.05, ***=p<0.001. (B) TCA cycle flux in Caco2 cells in which the expression of ALAS1 was down-regulated using two specific shRNAs (ALAS1 and ALAS1₂) compared to cells expressing a scramble shRNA. Results are expressed as pmol CO₂/h/mg protein. Data represent mean±SEM, n=3. For statistical analyses, an unpaired Student's t-test was used; *=p<0.05, **=p<0.01. (C) TCA cycle flux in Caco2 cells overexpressing FLVCR1a (indicated as FLVCR1a+) are compared to cells stably transduced with an empty vector. Moreover, the TCA cycle flux of FLVCR1a-overexpressing Caco2 cells treated with 25 μM hemin or 0.5 mM SA for 2 hours is reported. Results are expressed as pmol CO₂/h/mg protein. Data represent mean±SEM, n=3. For statistical analyses, an unpaired Student's t-test was used to compare the two matched groups; *=p<0.05, **=p<0.01. SA, succinylacetone.

FIG. 6. Relevance of the heme synthesis-export system in tumors.

(A) Frequency (%) of patient tumors with overexpression of FLVCR1 compared to their matched normal tissues across multiple cancer types. The number of patients showing tumor FLVCR1 overexpression relative to the total number of patients examined are indicated in the plot. Data derive from the BioXpress (Wan et al., 2015). For statistical analyses, a binomial test was used, the null hypothesis being equal probability of FLVCR1 being up or down-regulated in cancer for each patient. A P value of less than 0.01 was regarded as significant; *=p<0.01 (in all significant cases where the number of patients with FLVCR1 overexpression is significantly higher than expected in the null hypothesis), #=p<0.01 (in all significant cases where the number of patients with FLVCR1 overexpression is significantly lower than expected in the null hypothesis). (B, D) qRT-PCR analysis of the expression of FLVCR1a in SHSY-5Y cells (B) and BTECs (D) in which the expression of FLVCR1a was down-regulated using a specific shRNA, compared to cells expressing a scramble shRNA. Transcript abundance, normalized to beta-actin mRNA expression (for SHSY-5Y cells) or to 18s mRNA (for BTECs), is expressed as a fold increase over a calibrator sample. Data represent mean±SEM, n=4. For statistical analyses, an unpaired Student's t-test was used; ***p<0.001. (C, E) Western blot analysis of FLVCR1a expression in SHSY-5Y cells (C) and BTECs (E). Vinculin expression is shown as a loading control. Band intensities were measured by densitometry and normalized to vinculin expression. Densitometry data represent mean±SEM, n=2. For statistical analyses, an unpaired Student's t-test was used; *=p<0.05, **=p<0.01. Data are representative of three independent experiments. (F) Heme content in SHSY-5Y cells in which the expression of FLVCR1a was down-regulated using a specific shRNA, compared to cells expressing a scramble shRNA. Heme biosynthesis was stimulated with 5 mM 5-ALA for 15 hours. Values are expressed as pmol heme/mg protein. Data represent mean±SEM, n=3; *=p<0.05, **=p<0.01. (G) Heme content in BTECs in which the expression of FLVCR1a was down-regulated using a specific shRNA, compared to cells expressing a scramble shRNA. Values are expressed as pmol heme/mg protein. Data represent mean±SEM, n=6; **=p<0.01.

FIG. 7. Relevance of the heme synthesis-export system in vivo.

(A) Activities of the mitochondrial electron transport chain complexes I-IV in SHSY-5Y cells in which the expression of FLVCR1a was down-regulated using a specific shRNA, compared to cells expressing a scramble shRNA. Results were expressed as nmol NAD*/min/mg mitochondrial protein for complex I, nmol reduced cytochrome c/min/mg mitochondrial protein for complexes II-III, nmol oxidized cytochrome c/min/mg mitochondrial protein for complex IV. Data represent mean±SEM, n=3. For statistical analyses, an unpaired Student's t-test was used; *=p<0.05, **=p<0.01, *=p<0.001. (B) Mitochondrial ATP levels measured by a bioluminescent assay kit in SHSY-5Y cells in which the expression of FLVCR1a was down-regulated using a specific shRNA, compared to cells expressing a scramble shRNA. Results are expressed as nmol/mg mitochondrial proteins. Data represent mean±SEM, n=3. For statistical analyses, an unpaired Student's t-test was used; *=p<0.05. (C) TCA cycle flux in SHSY-5Y cells in which the expression of FLVCR1a was down-regulated using a specific shRNA, compared to cells expressing a scramble shRNA. Results are expressed as pmol CO₂/h/mg protein. Data represent mean±SEM, n=3. For statistical analyses, an unpaired Student's t-test was used; **=p<0.01. (D) Activities of the mitochondrial electron transport chain complexes I-IV in BTECs in which the expression of FLVCR1a was down-regulated using a specific shRNA, compared to cells expressing a scramble shRNA. Results were expressed as nmol NAD*/min/mg mitochondrial protein for complex I, nmol reduced cytochrome c/min/mg mitochondrial protein for complexes II-III, nmol oxidized cytochrome c/min/mg mitochondrial protein for complex IV. Data represent mean±SEM, n=3. For statistical analyses, an unpaired Student's t-test was used; **=p<0.01, *=p<0.001. (E) Mitochondrial ATP levels measured by a bioluminescent assay kit in BTECs in which the expression of FLVCR1a was down-regulated using a specific shRNA, compared to cells expressing a scramble shRNA. Results are expressed as nmol/mg mitochondrial proteins. Data represent mean±SEM, n=3. For statistical analyses, an unpaired Student's t-test was used; *=p<0.05. (F) TCA cycle flux in BTECs in which the expression of FLVCR1a was down-regulated using a specific shRNA, compared to cells expressing a scramble shRNA. Results are expressed as pmol CO₂/h/mg protein. Data represent mean±SEM, n=3. For statistical analyses, an unpaired Student's t-test was used; **=p<0.01. (G) Polymerase chain reaction (PCR) on Flvcr1a^{fl/fl};Cdh5-Cre^{ERT2} mice and control Flvcr1a^{fl/fl} mice following tamoxifen injection. Specific primers allowed the distinction of the wt (242 bp), floxed (280 bp) and null allele (320 bp) of Flvcr1a. Deletion of the first exon of Flvcr1a gene mediated by CRE recombinase gives rise to a band referred to as “null allele”. (H) TCA cycle enzymes activities are reported in boxes and expressed as mU/mg mitochondrial proteins. TECs isolated by subcutaneous tumors in tamoxifen-inducible endothelial specific Flvcr1a-null mice (Flvcr1a^{fl/fl};Cdh5-Cre^{ERT2} indicated as Flvcr1a-KO) are compared to TECs isolated by subcutaneous tumors in control Flvcr1a^{fl/fl} mice. Data represent mean±SEM, n=2 pools of animals. For statistical analyses, an unpaired Student's t-test was used; *=p<0.05. TECs, tumor endothelial cells. (I) Activities of the mitochondrial electron transport chain complexes I-IV in TECs isolated by subcutaneous tumors in tamoxifen-inducible endothelial specific Flvcr1a-null mice (Flvcr1a^{fl/fl};Cdh5-Cre^{ERT2} indicated as Flvcr1a-KO) are compared to TECs isolated by subcutaneous tumors in control Flvcr1a^{fl/fl} mice. Results were expressed as nmol NAD⁺/min/mg mitochondrial protein for complex I, nmol reduced cytochrome c/min/mg mitochondrial protein for complexes II-III, nmol oxidized cytochrome c/min/mg mitochondrial protein for complex IV. Data represent mean±SEM, n=2 pools of animals. For statistical analyses, an unpaired Student's t-test was used; *=p<0.05, **=p<0.01. TECs, tumor endothelial cells. (J) Mitochondrial ATP levels measured by a bioluminescent assay kit in TECs isolated by subcutaneous tumors in tamoxifen-inducible endothelial specific Flvcr1a-null mice (Flvcr1a^{fl/fl};Cdh5-Cre^{ERT2} indicated as Flvcr1a-KO) are compared to TECs isolated by subcutaneous tumors in control Flvcr1a^{fl/fl} mice. Results are expressed as nmol/mg mitochondrial proteins. Data represent mean±SEM, n=2 pools of animals. For statistical analyses, an unpaired Student's t-test was used; *=p<0.05. TECs, tumor endothelial cells.

FIG. 8. FLVCR1a silencing impairs cell survival and proliferation.

(A) Proliferation of Caco2 cells assessed by crystal violet staining at the indicated days; bar=1000 μm. Cells in which the expression of FLVCR1a was down-regulated using a specific shRNA are compared to cells expressing a scramble shRNA. Staining quantification is reported in the bar graph as percentage of crystal violet stained area. Data represent mean±SEM, n=4. For statistical analyses, a two-way analysis of variance was used, followed by the Bonferroni correction for multiple group comparisons; *=p<0.05, ***=p<0.001. (B) Caco2 cells viability the day of plating (day 0) and three days after plaiting (day 3). The measure is considered a readout of cell proliferation. Cells in which the expression of FLVCR1a was down-regulated using a specific shRNA are compared to cells expressing a scramble shRNA. Data represent mean±SEM, n=3 wells. For statistical analyses, a two-way analysis of variance was used, followed by the Bonferroni correction for multiple group comparisons; *=p<0.05, **=p<0.01. (C, D, E) Representative flow cytometric analyses of apoptosis in synchronized Caco2 (C), SKCO1 (D) and C80 (E) cells. Cells in which the expression of FLVCR1a was down-regulated using a specific shRNA are compared to cells expressing a scramble shRNA. The graph shows the percentage of apoptotic cells with respect to the entire population analyzed. Data represent mean±SEM, n=4 wells pooled from two independent experiments. For statistical analyses, an unpaired Student's t-test was used; *=p<0.05, **=p<0.01. (F) Tumor xenografts in NSG mice subcutaneously injected with SKCO1 cells in which the expression of FLVCR1a was down-regulated using a specific shRNA or with SKCO1 cells expressing a scramble shRNA. Representative tumor macroscopic images are shown (left panel). Moreover, representative hematoxylin and eosin stained sections of the tumors collected from mice injected with control cells (i) and FLVCR1a-silenced cells (ii) are shown (middle panels); bar=100 μm. Phenotypically, the two experimental groups show the same histopathological features, in particular a metaplastic trend of cancer cells toward cartilage or mature bone formation. The tumors from FLVCR1a-silenced cells display a higher amount of stroma (mostly fibrous tissue) and, in general, lower cellularity as compared to the tumors from control cells. Tumor volumes are reported in the dot plot (right panel). Data represent mean±SEM, n=6. For statistical analyses, an unpaired Student's t-test was used; *=p<0.05.

FIG. 9. FLVCR1a silencing reduces tumor endothelial cells proliferation and angiogenic potential in vitro.

(A) BTECs counting, as a readout of cell proliferation, at the indicated time points. Cells in which the expression of FLVCR1a was down-regulated using a specific shRNA are compared to cells expressing a scramble shRNA. Data represent mean±SEM, n=4. For statistical analyses, a two-way analysis of variance was used, followed by the Bonferroni correction for multiple group comparisons; ***p<0.001. (B) Quantification of BTECs migration rate in wound-healing assay. Cells in which the expression of FLVCR1a was down-regulated using a specific shRNA are compared to cells expressing a scramble shRNA. Data represent mean±SEM, n=32. For statistical analyses, a nonparametric unpaired Mann-Whitney test was used; ***p<0.001. (C) In vitro tubulogenesis assay on matrigel. BTECs in which the expression of FLVCR1a was down-regulated using a specific shRNA are compared to BTECs expressing a scramble shRNA. The number of nodes counted within the neo-formed vascular networks is shown. Data represent mean±SEM; n=10. For statistical analyses, a nonparametric unpaired Mann-Whitney test was used; **p<0.01.

FIG. 10. ALA treatment impairs the heme-synthesis export system and counteracts tumor cell survival/proliferation.

(A) Heme content in SHSY5Y cells non-treated (NT) or treated with 5 mM ALA for 15 hours. Values are expressed as pmol heme/mg protein. Data represent mean±SEM, n=3; *=P<0.05. (B, C) qRT-PCR analysis of the expression of ALAS1 (B) and FLVCR1a (C) in SHSY5Y cells non-treated (NT) or treated with 5 mM ALA for 15 hours. Transcript abundance, normalized to beta-actin mRNA expression, is expressed as a fold increase over a calibrator sample. Data represent mean±SEM, n=4. For statistical analyses, an unpaired Student's t-test was used; *p<0.05. (D) qRT-PCR analysis of the expression of ALAS1 in SKCO1 cells untreated (NT) or treated with 5 mM ALA for 24 hours. Transcript abundance, normalized to beta-actin mRNA expression, is expressed as a fold increase over a calibrator sample. Data represent mean±SEM, n=5. For statistical analyses, an unpaired Student's t-test was used; **p<0.01. (E) Western blot analysis of ALAS1 expression in SKCO1 cells untreated or treated with 5 mM ALA for 16, 24 and 48 hours. Beta-actin expression is shown as a loading control. Band intensities were measured by densitometry and normalized to beta-actin expression. Densitometry data represent mean±SEM, n=2. For statistical analyses, a one-way ANOVA analysis of variance was performed, followed by the Bonferroni correction for multiple groups comparisons; *=p<0.05, **=p<0.01. (F) Proliferation of SHSY5Y cells assessed by crystal violet staining at different time points (starting from the day after plating, named “day 0”). Treatment with 5 mM ALA occurred at “day 0” and cells were monitored until the end of the experiment without medium refresh. The proliferation of scramble-shRNA expressing cells, treated or not with ALA, is compared to that of non-treated FLVCR1a-silenced cells. The fold increase of the stained area relative to the correspondent stained area at “day 0” is shown. Data represent mean±SEM, n=3 wells for each time point. For statistical analyses, a two-way analysis of variance was used, followed by the Bonferroni correction for multiple group comparisons; ***=p<0.001. (G) SKCO1 cells viability measured by the CellTiter-Fluor Cell Viability Assay (Promega, Madison, WI USA, catalog n°G6080) at different time points, starting from the day after plating (named “day 0”), as a readout of cell proliferation. Untreated cells are compared to cells treated with 5 mM ALA at “day 0” and monitored until the end of the experiment without medium refresh. Cell viability is measured as fold increase over an untreated calibrator sample and expressed relative to day 0. Data represent mean±SEM, n=5 wells. For statistical analyses, a two-way analysis of variance was used, followed by the Bonferroni correction for multiple group comparisons; ***=p<0.001. (H) Flow cytometric (FACS) analyses of apoptosis in SHSY5Y cells treated with 5 mM ALA for 72 hours. The graph shows the percentage of apoptotic cells with respect to the entire population analysed. Data represent mean±SEM, n=2 wells. For statistical analyses, an unpaired Student's t-test was used; **=p<0.01. (I) LLC cell viability measured by the CellTiter-Fluor Cell Viability Assay (Promega, Madison, WI USA, catalog n°G6080) at different time points, starting from the day after plating (named “day 0”), as a readout of cell proliferation. Untreated cells are compared to cells treated with 0.5 mM SA or 5 mM ALA at “day 0” and monitored until the end of the experiment without medium refresh. Data represent mean±SEM, n=7 wells for each experimental point. For statistical analyses, a two-way analysis of variance was used, followed by the Bonferroni correction for multiple group comparisons; ***=p<0.001.

FIG. 11. FLVCR1a overexpression.

(A) pLVX-Puro-FLVCR1aMYC vector map. The human sequence for FLVCR1a fused in-frame with the human sequence for the MYC tag, named “FLVCR1aMYC sequence”. The “FLVCR1aMYC sequence” is inserted between EcoRI and SpeI enzyme restriction sites. The codified protein will contain the MYC-tag at the C-terminal of FLVCR1a. (B) The FLVCR1aMYC insert sequence, wherein: gaattc=EcoRI site; actagt=SpeI site; tga=stop codon; gaacaaaaactcateteagaagaggatctg=MYC-tag.

FIG. 12. FLVCR1a silencing.

pLKO.1 vector map. The relevant components are indicated at the top right of the figure. The mature antisense sequence originating from the shRNA, and targeting the FLVCR1a mRNA, is reported below. The complementary sense sequence will be degraded by host cells, according to the standard progress of the RNA-interference process.

FIG. 13. ALAS1 silencing.

pLKO.1 vector map. The relevant components are indicated at the top right of the figure. The mature antisense sequence originating from the shRNA, and targeting the ALAS1 mRNA, is reported below. Two different shRNAs (codified by separate vectors) have been chosen, both targeting ALAS1 mRNA, but in different sequence tracts. The correspondent complementary sense sequence will be degraded by host cells, according to the standard progress of the RNA-interference process.

### DETAILED DESCRIPTION OF THE INVENTION

In the description that follows, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

As used herein, the term “blocker” means a substance which prevents or inhibits a given physiological function. In the present case the blocker prevents normal functioning of the heme synthesis-export axis by inhibiting the synthesis of heme within the cell (through feedback inhibition of ALAS1 enzyme and/or silencing of ALAS1 enzyme) and/or down-modulating the export of heme outside the cell (through silencing of FLVCR1a heme exporter).

As used herein, the expression “active agent” means a substance or combination of substances used in a finished pharmaceutical product, intended to furnish pharmacological activity or to otherwise have direct effect in the cure, mitigation, treatment or prevention of disease, or to have direct effect in restoring, correcting or modifying physiological functions in human beings. It is excluded from such a definition an agent that is used as adjuvant or sensitizer, i.e. a substance, or a combination of substances, employed to enhance the effectiveness of a medical treatment (e.g. 5-aminolevulinic acid used as sensitizer in the photodynamic therapy). It will thus be appreciated that the expression “active agent” means a substance or combination of substances that is not used as adjuvant or sensitizer of a medical treatment which may comprise applying energy (e.g. heat, ultrasounds, light, etc.) to a patient.

As used herein, the expression “feedback inhibitor” means a substance that suppresses the activity of an enzyme, participating in a sequence of reactions by which the substance is synthesized, by an end-product of that sequence. When the end-product accumulates in a cell beyond an optimal amount, its production is decreased by inhibition of an enzyme involved in its synthesis. After the end-product has been utilized or broken down and its concentration thus decreased, the inhibition is relaxed, and the formation of the end-product resumes. In the present case 5-aminolevulinic acid, derivatives and salts thereof determine heme accumulation inside the cell with feedback inhibition of ALAS1 enzyme, which is prevented from participating in heme synthesis.

As used herein, the term “down-regulator” means a substance able to reduce or suppress a response to a stimulus. In the present case the down-regulator is a substance able to reduce expression of FLVCR1a heme exporter or ALAS1 enzyme; preferably the down-regulator is represented by shRNAs able to silence FLVCR1a or ALAS1 (i.e. down-modulating their expression) and, as a consequence, preventing heme export by FLVCR1a outside the cell or heme synthesis by ALAS1 enzyme.

As used herein, the expression “ALA derivative” means for example a derivative comprising an ester group and/or acyl group of ALA. Preferably, a combination of methylester group and formyl group; methylester group and acetyl group; methylester group and n-propanoyl group; methylester group and n-butanoyl group; ethylester group and formyl group; ethylester group and acetyl group; ethylester group and n-propanoyl group; ethylester group and n-butanoyl group can be exemplified.

As used herein, the expression “ALA salt” means acid addition salts (such as hydrochloride, hydrobromide, hydroiodide, phosphate, nitrate, sulfate, acetate, propionate, toluenesulfonate, succinate, oxylate, lactate, tartate, glycolate, methanesulfonate, butyrate, valerate, citrate, fumarate, maleate, and malate); metal salts (such as sodium salt, potassium salt, and calcium salt); ammonium salt, and alkyl ammonium salt. These salts may form a hydrate or solvate, and can be used separately, or by combining two or more of them.

As used herein, the expression “shRNA analogue of SEQ ID No.: x” means a sense/antisense strand RNA comprising a base sequence wherein one to several bases have been added to and/or deleted from the 5′ terminal and/or 3′ terminal of the base sequence described in SEQ ID No.: x, and which optionally has an overhang at the terminal of the sense/antisense strand.

In one embodiment, the present invention concerns a blocker of heme synthesis-export axis as active agent for use in the treatment of a tumor in a subject, wherein the blocker of heme synthesis-export axis upmodulates oxidative metabolism of tumor cells by upregulating the tricarboxylic acid (TCA) cycle and the oxidative phosphorylation (OXPHOS).

In one embodiment, the blocker of heme synthesis-export axis exerts antiproliferative and proapoptotic effects on tumor cells.

In one embodiment, the tumor treatment comprises a reduction or inhibition of tumor proliferation, tumor angiogenesis, and/or tumor pain.

In one embodiment, the blocker of heme synthesis-export axis is selected from (i) a feedback inhibitor of ALAS1 enzyme, (ii) a down-regulator of FLVCR1a heme exporter and (iii) a down-regulator of ALAS1 enzyme.

In one embodiment, the feedback inhibitor of ALAS1 enzyme is selected from 5-aminolevulinic acid, derivatives and salts thereof, preferably the feedback inhibitor of ALAS1 enzyme is 5-aminolevulinic acid.

In one embodiment, the down-regulator of FLVCR1a heme exporter is selected from shRNAs complementary to the FLVCR1a mRNA sequence set forth in SEQ ID No.: 1 (corresponding to sequence NM_014053.4 Homo sapiens FLVCR heme transporter 1 (FLVCR1), mRNA), wherein the shRNAs comprise an antisense strand of 19-25 continuous bases, preferably 19-22 continuous bases, and a matching sense strand.

In one embodiment, the down-regulator of FLVCR1a heme exporter is selected from a shRNA comprising an antisense strand as set forth in SEQ ID No.: 2 and a matching sense strand and analogues thereof. Preferably, the down-regulator of FLVCR1a heme exporter is a shRNA having an antisense strand as set forth in SEQ ID No.: 2 and a matching sense strand.

In one embodiment, the down-regulator of ALAS1 enzyme is selected from shRNAs complementary to the ALAS1 mRNA sequence set forth in SEQ ID. No.: 15 (corresponding to sequence NM_000688.6 Homo sapiens 5′-aminolevulinate synthase 1 (ALAS1), transcript variant 1, mRNA; nuclear gene for mitochondrial product), wherein the shRNAs comprise an antisense strand of 19-25 continuous bases, preferably 19-22 continuous bases, and a matching sense strand.

In one embodiment, the down-regulator of ALAS1 enzyme is selected from a shRNA comprising an antisense strand as set forth in SEQ ID No.: 10 and 12 and a matching sense strand and analogues thereof.

In one embodiment, the blocker of heme synthesis-export axis is used as a single active agent or in combination with a different anti-tumor agent, provided that 5-aminolevulinic acid is not used in combination with artemisinin, artesunate, HDACs inhibitors, sodium ferrous and methadone. Preferably, the blocker of heme synthesis-export axis is used as a single active agent.

In one embodiment, the blocker of heme synthesis-export axis is administered by intra-arterial/intracavitary/intraperitoneal/intrapleural/intrathecal infusion, intravascular/intramuscular injection, intra-tumor injection, inhalation, intratracheal/conjunctival/ear/laryngeal/nasal/bladder/urethral instillation, topical/transdermal application, subcutaneous/oral/rectal administration.

In one embodiment, the feedback inhibitor or the down-regulator of ALAS1 enzyme and the down-regulator of FLVCR1a heme exporter are administered together in a combination therapy.

In one embodiment, the feedback inhibitor of ALAS1 enzyme is administered together with at least one iron compound. Preferably the at least one iron compound is selected from ferrous citrate, ferric citrate, ferric ammonium citrate, ferric pyrophosphate, dextran iron, ferric lactate, ferrous gluconate, DTPA Iron, ammonium iron ethylenediaminetetraacetate, ferric ammonium ethylenediaminetetraacetate, triethylenetetramine iron, dicarboxylic acid iron, ferric chloride, ferric iron, ferric chloride, ferric oxide, ferritin, ferrous fumarate, ferrous pyrophosphate, iron-containing iron oxide, iron acetate, iron oxalate, ferrous succinate, ferric sulfate and sulfur acid iron.

In one embodiment, the present invention concerns a pharmaceutical composition comprising as active agent a blocker of heme synthesis-export axis as defined above for use in the treatment of a tumor in a subject.

The results described herein show that enhanced heme export is required to sustain heme synthesis and illustrate the functionality of a heme synthesis-export axis adopted by cells to reduce the TCA cycle flux, with the ensuing reduction of OXPHOS. Thus, the present data demonstrate that the heme synthesis-export system is exploited by both tumor cells and TECs to down-modulate oxidative metabolism, thus contributing to metabolic rewiring required for tumor initiation and progression. Moreover, the present work provides evidence that disruption of this system by both heme export down-modulation (through a specific FLVCR1a shRNA) and heme synthesis inhibition (by using ALA or specific ALAS1 shRNAs) results in reduced cancer cells and tumor endothelial cells survival and proliferation.

The importance of FLVCR1a in cancer has previously been reported in synovial sarcoma (Peng et al., 2018) and hepatocellular carcinoma (Shen et al., 2018), and the relevance of heme synthesis for cancer is known. Moreover, the relationship between heme synthesis and heme export was already described in the liver, where the system ensures the supply of heme to meet requirements for the activity of cytochromes like cytochromes P450 (Vinchi et al., 2014). However, to inventors' knowledge, there are not previous literature data clearly showing a reciprocal regulation of the two processes (heme synthesis and heme export) in the context of cancer and of tumor angiogenesis, as well as a role for FLVCR1a and the heme synthesis-export system in the regulation of the TCA cycle flux and OXPHOS.

Although, based on the present data, the heme synthesis-export axis is an important system for cancer cell survival/proliferation, the mechanism has never been explored by the scientific community so far, likely because sustaining heme synthesis by promoting heme export is a counterintuitive concept. Indeed, it implies that cells consume energy to produce heme, but then throw part of it away. Likewise, the observed enhancement of heme synthesis associated with reduced activity of ETC complexes, which exploit heme as a co-factor, is also counterintuitive. Finally, the present data are in apparent contrast with other studies showing that the administration of exogenous heme promotes ETC activity (Vandekeere et al., 2018). Nevertheless, the present inventors think that the mechanism they identified can reconcile these apparent contradictions. Indeed, results from several studies have documented that ALAS1, the rate limiting enzyme of the heme biosynthetic pathway, is negatively regulated by heme itself. Heme controls transcription, translation, and stability of ALAS1 mRNA. Moreover, heme binding to heme regulatory motifs, situated in the mitochondrial targeting sequence of ALAS1, inhibits protein translocation to mitochondria. The present inventors found that FLVCR1a-mediated heme export introduces a further level of regulation by controlling the size of the regulatory heme pool responsible for ALAS1 modulation. The present data also demonstrate that heme efflux finely tunes the rate of heme synthesis required for the regulation of the TCA cycle flux, which in turn sustains OXPHOS. The modulation of the TCA cycle by the heme synthesis-export axis might be explained by ALAS1-mediated TCA cycle cataplerosis. Moreover, literature data show heme-dependent control of pyruvate dehydrogenase (PDH), a key enzyme for the supply of TCA cycle by glucose and of the transcription factor BTB and CNC homology 1 (BACH1), that modulates the expression of some glycolytic enzymes and the activity of PDH. Thus, the induction of FLVCR1a expression represents a strategy adopted by cells to shut down oxidative metabolism, while ensuring adequate heme supply for hemoproteins' production. Finally, the inventors' finding that the administration of exogenous heme phenocopied FLVCR1a silencing, leading to increased TCA cycle flux that in turn supports the activity of ETC complexes, provides an alternative explanation for the seemingly discrepant observations reported by (Vandekeere et al., 2018).

The TCA cycle is a central hub for cell energy metabolism, the synthesis of macromolecules, and redox balance. Impaired TCA cycle functions are associated with a wide variety of pathological processes, encompassing cancer, obesity, neurodegenerative disorders, infections, muscular diseases, diabetes etc. Several components or indirect modulators of the TCA cycle may be exploited for therapeutic purposes and, although high toxicity remains an issue, some of these approaches have proven to be well tolerated clinically. The present data identified the heme synthesis-export axis as a potentially targetable vulnerability to modulate the TCA cycle flux. The present inventors focused on two main approaches to disrupt the heme synthesis-export system: the down-modulation of FLVCR1a by a specific shRNA and the reduction of heme synthesis by the administration of ALA or the down-modulation of ALAS1 by specific shRNAs.

The present inventors chose FLVCR1a as a target because it is a plasma membrane protein, that makes it a good candidate for the development of specific inhibitors/activators particularly in the context of cancer, where this heme exporter is overexpressed compared to normal tissues. The present data demonstrate that the silencing of FLVCR1a by a specific shRNA is effective in reducing both cancer cells and tumor endothelial cells survival and proliferation. In the case of endothelial cells, the present inventors also obtained in vitro evidence that blunting FLVCR1a-mediated heme export leads to alteration of angiogenic properties, thus impairing not only tumor growth but also tumor vascularization.

As an alternative or complementary approach, the present inventors propose the targeting of ALAS1 by specific shRNAs. Indeed, the present data show that the down-modulation of ALAS1 induces metabolic effects comparable to those obtained by FLVCR1a silencing.

In addition, to reduce ALAS1 activity, the present inventors also tested the use ALA. Following the pioneer work by Malik (Malik and Lugaci, 1987), Kennedy and Pottier (Kennedy et al., 1990) and Moan and Peng (Peng et al., 1992) who showed enhanced ALA-mediated protoporphyrin IX (PpIX) accumulation in tumor cells and effective cell destruction after light illumination, ALA was rapidly established as a promising PDT agent. With proven effectiveness in eliminating unwanted cells, good selectivity and excellent cosmetic effect, ALA-PDT received world-wide approval in the late 1990s and has become a mainstream treatment in dermatology. Its applications in managing other types of cancers and non-cancer diseases are being actively explored as well. Not only is it a remarkable PDT agent, ALA is also a useful imaging probe. With a broad red fluorescence emission extending close to the near-infrared region, ALA-mediated PpIX fluorescence is being used to guide the resection of brain and bladder tumors with encouraging clinical outcomes. The key to the successful use of ALA as a PDT and imaging agent lies in the preferential accumulation of PpIX in target cells following ALA administration. Extensive research has been performed to determine the molecular mechanism involved in enhanced ALA-PpIX in tumor cells compared with normal counterparts, which provides the basis for using ALA as a prodrug for fluorescence detection and photodynamic targeting of tumors. Although this remains an open question, extensive studies have suggested that increased PpIX fluorescence in tumor cells is likely a result of multiple tumor-associated cellular alterations including deregulations in heme biosynthetic enzymes, mitochondrial functions and porphyrin transporters.

Other than the use of ALA for ALA-PDT and tumor fluorescence-guided surgery (FGS), ALA was also exploited as a sensitizer in studies testing the use of other drugs for cancer, such as artemisinin (Wang et al., 2017), artesunate and HDACs inhibitors (Chen et al., 2019), or for other diseases, such as sodium ferrous (Wang et al., 2021) and methadone (Shi et al., 2019).

Thus, to the inventors' knowledge, all previous studies explored the use of ALA as a sensitizing agent in combination with PDT or other drugs for therapeutic purposes, or alone as a stimulator of heme biosynthesis in experimental protocols.

The present inventors considered that, by eliciting heme production bypassing ALAS1, prolonged ALA treatment could favour heme accumulation inside the cells, with the ensuing feedback inhibition of ALAS1. Thus, they tested the use ALA as a heme-synthesis inhibitor rather than a heme-synthesis stimulator and evaluated the ability of ALA to disrupt the heme synthesis-export system in order to compromise tumor cell survival and proliferation. The present data demonstrate that ALA treatment down-modulates the heme synthesis-export system and exerts antiproliferative/pro-apoptotic effects on tumor cells. These results support the use of ALA as an anticancer agent per se, and not as a sensitizer associated to PDT or other drugs. It will be appreciated that according to the present invention ALA is used as active agent (as defined above) not in combination with PDT or any medical treatment which comprises applying energy (e.g. light, heat, ultrasounds, etc.) to a patient. Moreover, considering the importance of the heme synthesis-export system in tumor endothelial cells, the use of ALA proposed limits abnormal tumor vascularization and enhance the effects of antiangiogenic agents. In order to potentiate and sustain over time the effects of ALA, the inventors also considered the possibility to combine ALA with iron, avoiding exhaustion of this metal required for the enzymatic reaction promoted by ferrochelatase (FECH), the last enzyme of heme biosynthesis.

Tumor development is a complex process that involves the cooperation of different environmental components. Besides the importance of the immune system and vascular development to sustain tumor growth, accumulating evidences support the concept that tumor innervation represents an additional crucial aspect in the promotion of tumor progression. Further, many tumor types are more densely innervated than their normal tissues of origin. Besides sustaining cancer growth, tumor innervation also contributes to cancer associated pain. Tumor innervation is supported by the secretion of neutrophins and axon guidance molecules by cancer cells that promotes neurite outgrowth. Remarkably, evidences suggests that the activity of neudesin, a neurotrophic factor overexpressed by cancer cells, is regulated by heme. Furthermore, the inhibition of heme synthesis impairs NGF signalling. Finally, mutations in the FLVCR1 gene have been associated with pain insensitivity in humans. Based on these evidences, the disruption of the heme synthesis-export system by the FLVCR1a specific shRNA, and/or the ALAS1 specific shRNAs or ALA is expected to interfere with tumor innervation and be beneficial to counteract tumor growth and cancer-associated pain.

Thus, by using specific FLVCR1a shRNA and/or ALA or specific ALAS1 shRNAs, the present inventors envision a combinatorial detrimental effect on cancer growth, tumor vascularization and tumor innervation.

In conclusion, the present work identifies the heme synthesis-export axis as a key regulator of the TCA cycle and oxidative metabolism and puts forth the targeting of this system by a specific shRNAs to FLVCR1a and ALAS1 and by the FDA-approved drug ALA, for which a new use was identified.

### Results

### The FLVCR1a-Mediated Heme Export Sustains Heme Synthesis.

Heme synthesis is mainly controlled by heme itself through a feedback inhibition on ALAS1. Therefore, the present inventors hypothesized that the promotion of heme export by FLVCR1a could be a strategy adopted by cells to avoid heme accumulation and in this way ensure sustained heme synthesis.

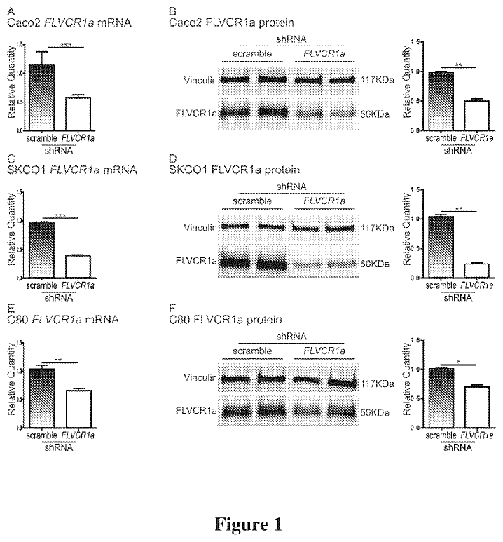

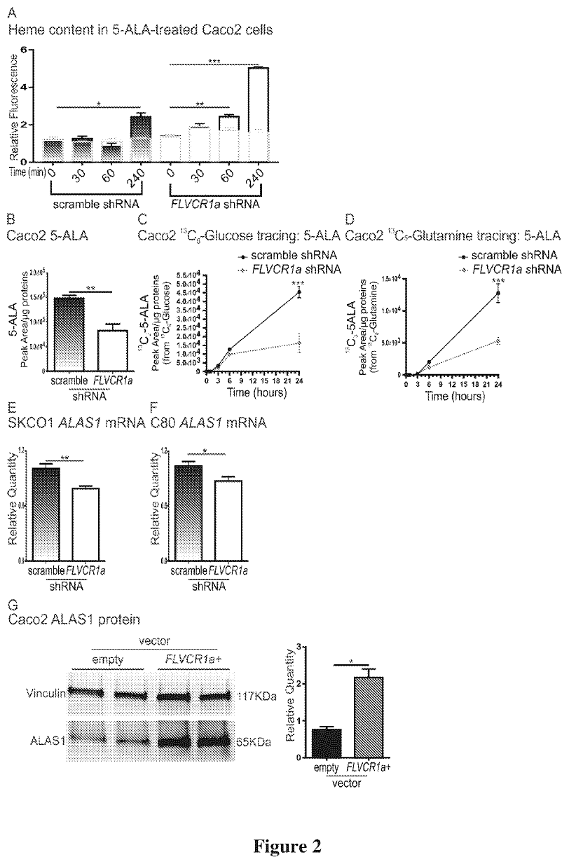

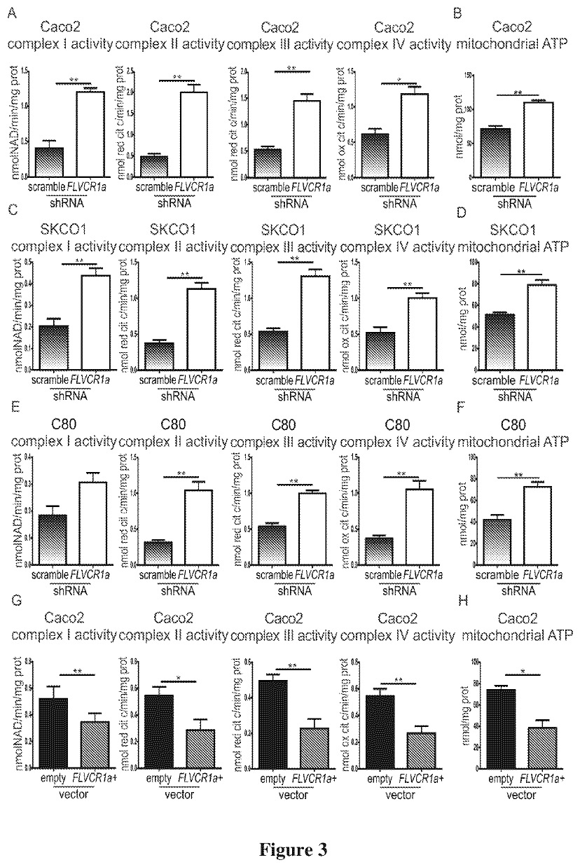

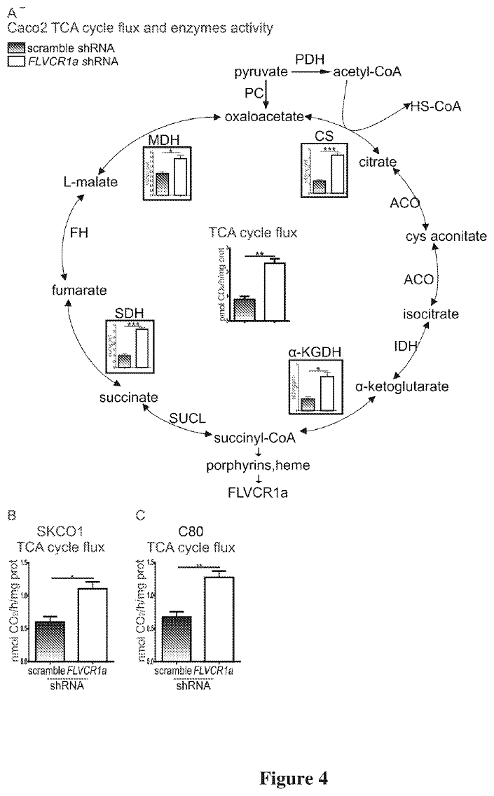

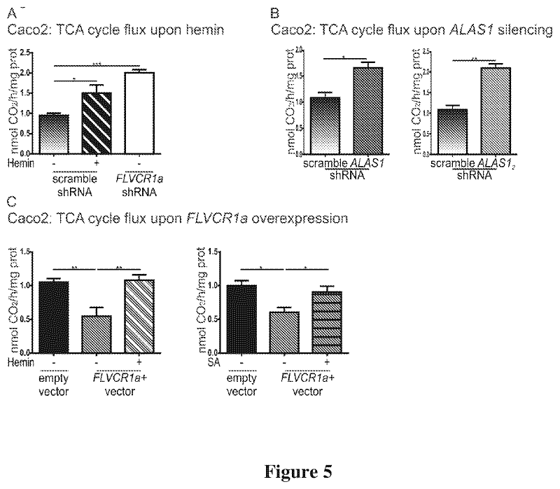

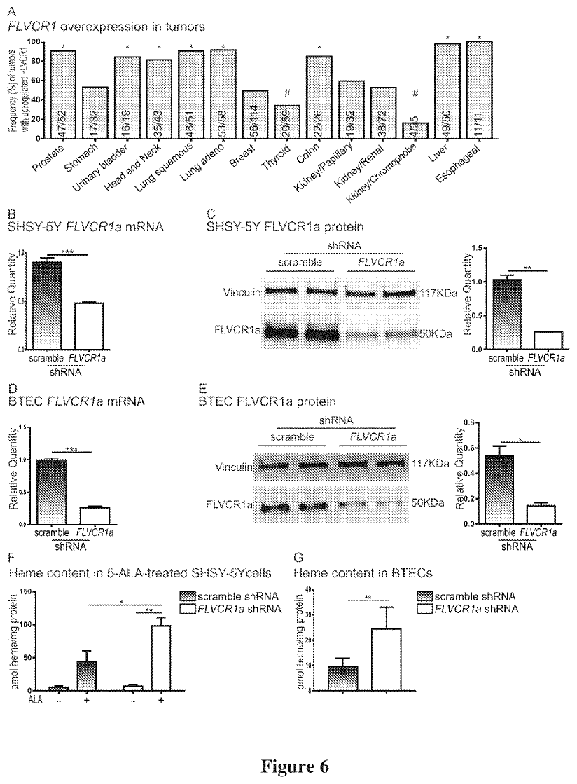

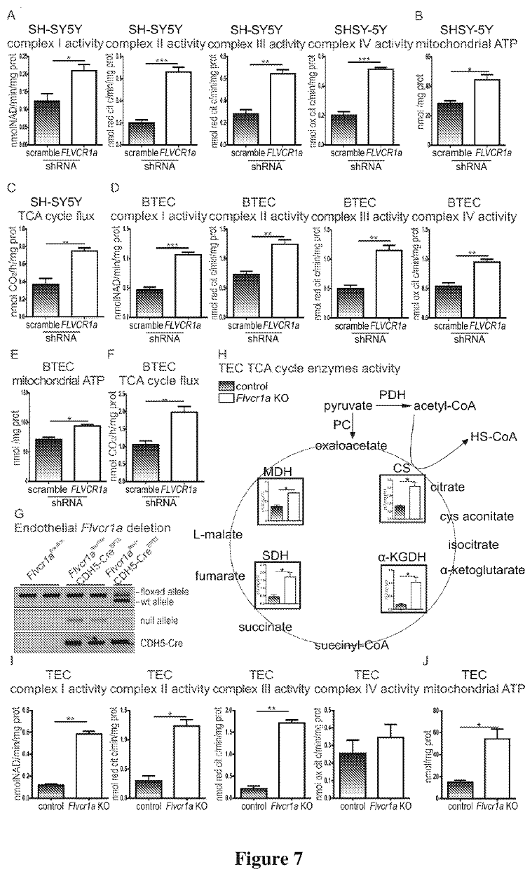

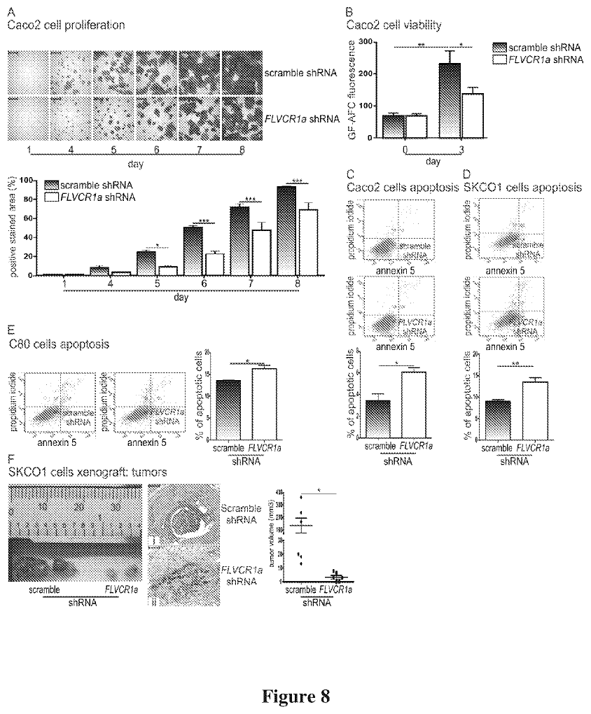

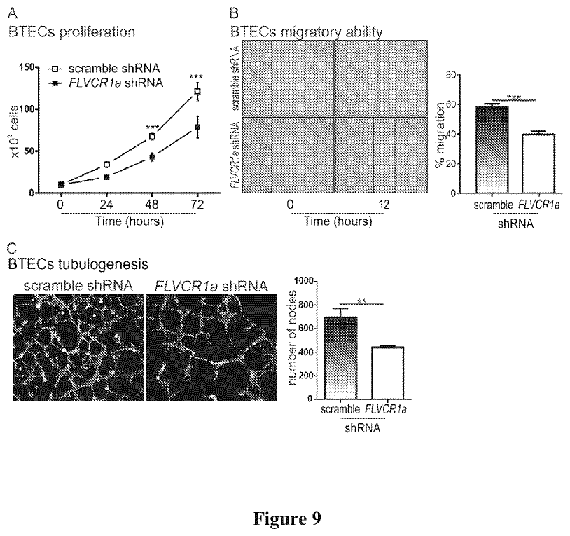

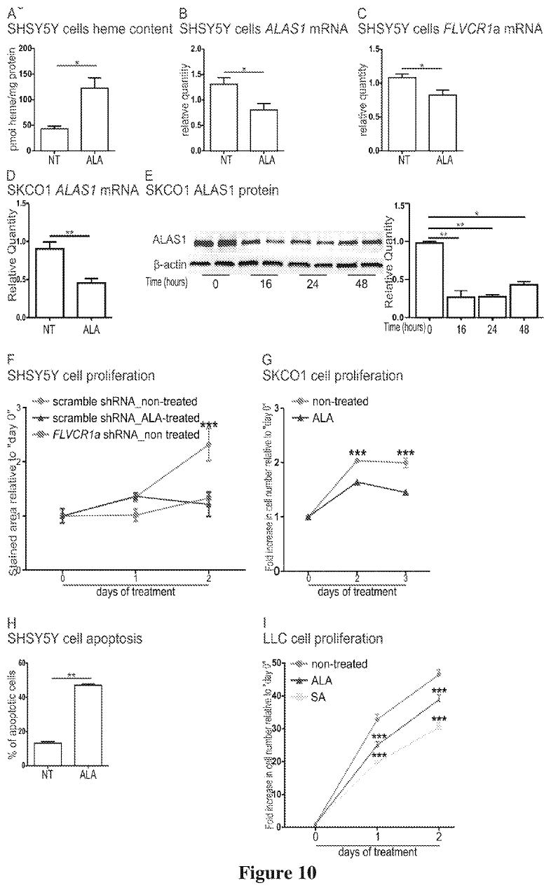

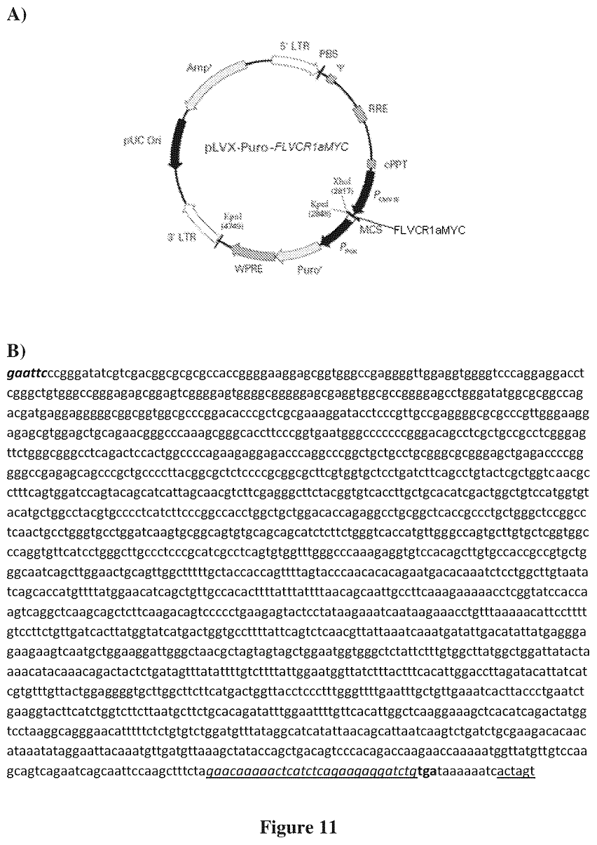

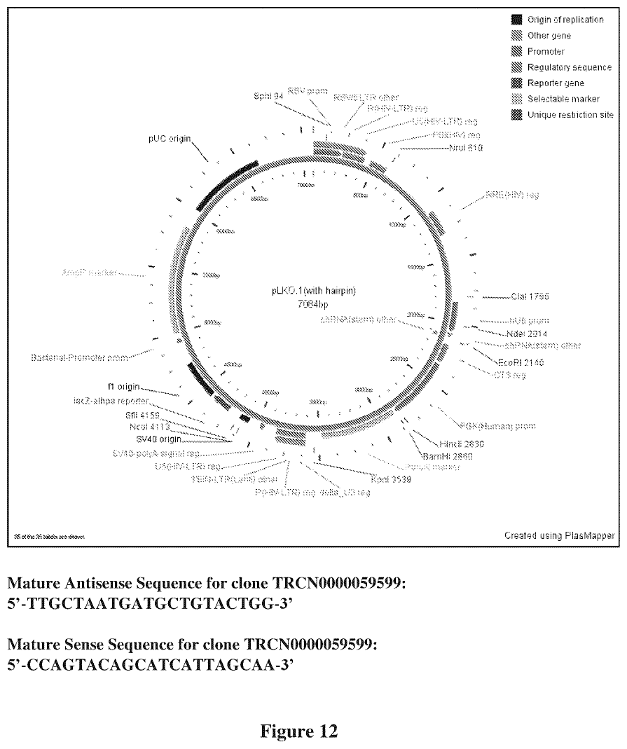

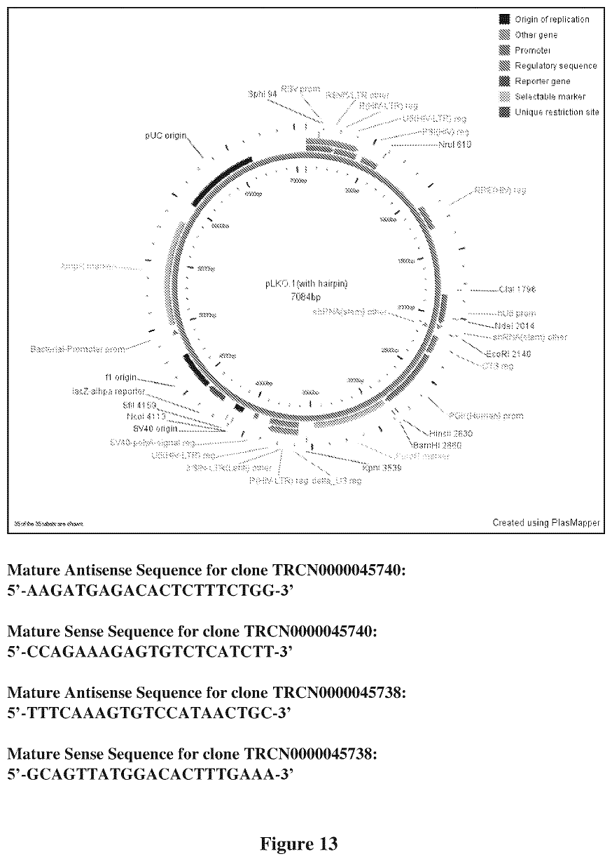

To test this hypothesis, the present inventors chose as model systems the colorectal cancer (CRC) cell lines Caco2, C80 and SKCO1, in which the inventors silenced FLVCR1a using RNA interference [FIG. 1].

First, the inventors analyzed whether FLVCR1a was involved in the export of de-novo synthesized heme in these cells. Upon induction of heme biosynthesis by the heme precursor 5-aminolevulinic acid (5-ALA), FLVCR1a-silenced cells experienced a faster and higher increase in the amount of intracellular heme than control cells, indicating that FLVCR1a participates to the export of newly produced heme [FIG. 2A]. This accumulation was blunted by the heme biosynthesis inhibitor succinylacetone (SA) [FIG. 2A], confirming that FLVCR1a prevents the accumulation of de-novo synthesized heme. The inventors also analyzed the rate of heme synthesis in FLVCR1a-silenced Caco2 cells, taking advantage of metabolomic and tracing analyses. Among heme precursors, only 5-ALA could be detected in the experimental conditions of the performed assays. However, 5-ALA synthesis is the rate-limiting step in heme biosynthesis, reflecting the progress of the entire process. By measuring the intracellular production of 5-ALA, the inventors observed a lower amount of 5-ALA in FLVCR1a-silenced Caco2 cells as compared to controls [FIG. 2B]. In addition, in time course experiments, FLVCR1a-deficient Caco2 cells maintained in medium with uniformly labelled glucose (U-¹³C-glucose) or glutamine (U-¹³C-glutamine) showed reduced incorporation of labelled carbons in 5-ALA [FIGS. 2C and 2D], confirming that ALAS1 activity was reduced in these cells relative to controls. These data strengthen the assumption that FLVCR1a function is required to sustain heme synthesis. Consistent with data obtained in Caco2 cells, the inventors observed reduced ALAS1 expression in FLVCR1a-silenced SKCO1 and C80 cells [FIGS. 2E and 2F]. Conversely, FLVCR1a overexpression in Caco2 cells resulted in increased ALAS1 expression [FIG. 2G].

These results indicate that, by prompting heme export, FLVCR1a limits the feedback inhibitory effect of accumulated heme on ALAS1, thus favoring heme production. Hence, the two processes of heme synthesis and heme export define a unique system, where heme export is crucial to sustain heme synthesis.

### The Heme Synthesis-Export System Controls the OXPHOS Rate and the TCA Cycle Flux.

Given the strong relationship between heme and cell energy production, the inventors sought to analyze the impact of heme synthesis-export axis on cellular energetic metabolism. To this end, they evaluated mitochondrial function in FLVCR1a-silenced cells.

FLVCR1a silencing resulted in increased activity of all the complexes involved in the mitochondrial respiratory chain [FIG. 3A], including ATP synthase [FIG. 3B]. Moreover, FLVCR1a-silenced cells showed significantly higher ATP levels in mitochondria [FIG. 3C] than control cells.

The increased ETC complexes activity and mitochondrial ATP levels observed in FLVCR1a-silenced Caco2 cells were confirmed in SKCO1 [FIGS. 3D and 3E] and C80 cells [FIGS. 3F and 3G]. In a complementary perspective, FLVCR1a-overexpressing cells showed the opposite phenotype, with a significant reduction in the activity of all the ETC complexes and of mitochondrial ATP levels [FIGS. 3H and 3I].

OXPHOS is sustained by the TCA cycle and heme synthesis participates in TCA cycle cataplerosis. Accordingly, the inventors examined metabolic differences in the TCA cycle in FLVCR1a-deficient versus FLVCR1a-proficient Caco2 cells. The total flux of the TCA cycle resulted significantly up-regulated in FLVCR1a-silenced cells compared to controls [FIG. 4A, see the histogram in the center of the figure], and several TCA cycle enzymes, including citrate synthase, α-ketoglutarate dehydrogenase, succinate dehydrogenase and malate dehydrogenase [FIG. 4A], displayed enhanced activity following FLVCR1a knockdown. Increased TCA cycle flux upon FLVCR1a silencing was confirmed in SKCO1 and C80 cells [FIGS. 4B and 4C].

The present data suggest that FLVCR1a controls the TCA cycle by modulating the intracellular heme pool involved in ALAS1 inhibition. Consistently, hemin administration in control Caco2 cells to induce feedback reduction of ALAS1 activity led to increased TCA cycle flux, mimicking the effects of decreased heme export in FLVCR1a-silenced cells [FIG. 5A]. Moreover, the inventors observed an increased TCA cycle flux in ALAS1-silenced Caco2 cells [FIG. 5B], similar to that reported for FLVCR1a-silenced cells. Conversely, FLVCR1a-overexpressing cells showed the opposite phenotype, with a significant reduction of the TCA cycle flux, which could be reverted by treatment with hemin or with the heme synthesis inhibitor succinylacetone (SA) [FIG. 5C]. Together, these findings strengthen the notion that FLVCR1a controls the TCA cycle flux by modulating the rate of heme synthesis.

The overall dataset indicates that FLVCR1a participates to the regulation of ALAS1 activity by modulating intracellular heme accumulation, with implications in the control of the TCA cycle flux and, consequently, on the rate of OXPHOS.

### Relevance of the Heme Synthesis-Export System In Vivo

Mining of the BioXpress database revealed overexpression of FLVCR1 in several tumor types other than CRC [FIG. 6A], suggesting that the ALAS1-FLVCR1a system could be exploited to modulate oxidative metabolism in different tumor contexts. To address this point, the inventors down-modulated FLVCR1a in neuroblastoma SH-SY5Y cells [FIGS. 6B and 6C] and in tumor endothelial cells (BTECs, Breast cancer-derived Tumor Endothelial Cells) [FIGS. 6D and E].

In SH-SY5Y cells, FLVCR1a silencing resulted in higher heme accumulation than controls upon stimulation of de-novo heme biosynthesis [FIG. 6F]; in BTECs, increased heme levels were already detectable at the steady state after FLVCR1a knockdown [FIG. 6G], indicating that also in these cell lines FLVCR1a down-modulation blocks heme efflux. Moreover, in both FLVCR1a-silenced cell lines, the inventors observed enhanced activity of ETC complexes with increased mitochondrial ATP levels [FIGS. 7A, 7B, 7D and 7E] as well as increased TCA cycle flux [FIGS. 7C and 7F], confirming the observations obtained in CRC cell lines.

Finally, to evaluate the in vivo relevance of the inventors' findings, they took advantage of tamoxifen-inducible endothelial specific Flvcr1a-null mice (Flvcr1a^{fl/fl};Cdh5-Cre^{ERT2}) [FIG. 7G] and analyzed oxidative metabolism in tumor endothelial cells (TECs) isolated from subcutaneous tumors. As observed in vitro, also in Flvcr1a-deleted endothelial cells (indicated as Flvcr1a-KO), the activity of TCA cycle enzymes [FIG. 7H], as well as that of ETC complexes and the levels of mitochondrial ATP [FIGS. 71 and 7J], were higher than in control counterparts. These data support the notion that FLVCR1a-mediated heme export modulates TCA cycle and OXPHOS.

### Functional Consequences of Impaired Heme Synthesis-Export

As illustrated above, the enhancement of heme synthesis associated to heme export in tumors contributes to the down-modulation of the TCA cycle and OXPHOS. Thus, the forced implementation of oxidative metabolism and the deregulation of the TCA cycle that occur upon FLVCR1a silencing could have consequences on tumor cell properties, including survival, growth and migratory capacity.

Therefore, the inventors soughed to assess the impact of compromised heme synthesis-export on tumor cells. To this end, they exploited two complementary strategies: FLVCR1a silencing, to blunt heme export, and 5-ALA treatment, to elicit heme accumulation with the ensuing feedback inhibition of ALAS1-dependent heme synthesis.

FLVCR1a-silenced Caco2 cells proliferated more slowly [FIGS. 8A and 8B] and displayed a higher extent of basal apoptosis [FIG. 8C] than control cells. Similar to Caco2 cells, FLVCR1a silencing led to increased apoptosis also in SKCO1 and C80 cells [FIGS. 8D and 8E]. In line with in vitro results, subcutaneous injection of FLVCR1a-silenced SKCO1 cells in NOD-SCID gamma (NSG) mice gave rise to smaller tumors than those obtained by the injection of control cells [FIG. 8F], confirming the inhibitory effect of FLVCR1a knockdown on CRC cell proliferation/survival also in an in vivo context.

Consistent with data obtained in CRC cells, the inventors observed reduced proliferation of FLVCR1a-silenced BTECs [FIG. 9A]. They also studied the effects of FLVCR1a-deficiency on the angiogenic potential of BTECs by performing in vitro wound-healing assay and tubulogenesis assay. FLVCR1a-silenced BTECs showed a reduction of the migratory rate [FIG. 9B] as well as an impaired ability to form a complex microvascular network on matrigel [FIG. 9C].

These data are in line with a documented involvement of FLVCR1a in cell survival and proliferation in different cell lines and tissues, including the physiological proliferation of intestinal mucosa cells (Fiorito et al., 2015) and normal endothelial cells (Petrillo et al., 2018), as well as the survival/proliferation of cells in the nervous system (Chiabrando et al., 2016, Bertino et al., 2019).

The inventors then tested the effects of 5-ALA. Experiments in neuroblastoma SHSY5Y cells showed that cell treatment with 5-ALA promoted heme accumulation inside the cells [FIG. 10A] and this, in turn, resulted in the inhibition of the heme synthesis-export axis, as indicated by reduced ALAS1 and FLVCR1a transcript levels [FIGS. 10B and 10C]. Similarly, 5-ALA administration for 16, 24 and 48 hours to the colorectal cancer SKCO1 cells resulted in decreased ALAS1 mRNA and protein levels [FIGS. 10D and 10E]. The treatment mimicked the effects of the genetic down-modulation of the axis obtained by FLVCR1a-silencing. Indeed, ALA treatment led to reduced SHSY5Y cell proliferation, reaching a rate comparable to that detected for FLVCR1a-silenced cells [FIG. 10F]. Reduced proliferation upon ALA treatment was also observed in SKCO1 cells [FIG. 10G]. Moreover, increased apoptosis was also observed in SHSY5Y upon ALA treatment [FIG. 10H].

Analogously, 5-ALA treatment significantly reduced lung tumor LLC cells proliferation [FIG. 10I]. The effects on cell proliferation were similar to those obtained with a known heme synthesis inhibitor, succynilacetone (SA), that targets ALAD (5-aminolevulinic acid dehydratase), the second enzyme of the heme biosynthetic pathway, bypassing ALAS1 [FIG. 10I].

Collectively, the results obtained provided evidence that the forced impairment of the heme synthesis-export system is deleterious for cancer cells survival/proliferation and, in case of tumor endothelial cells, also for the angiogenic properties in vitro.

### Materials and Methods

### Cell Lines

Caco2 cells (ATCC, Manassas, VA USA, catalog n° HTB-37, RRID:CVCL_0025) were maintained in Dulbecco's modified Eagle's medium (DMEM, high glucose, GlutaMAX supplement; Gibco by Thermofisher Scientific, Waltham, MA USA, catalog n°61965059) supplemented with 20% heat-inactivated low-endotoxin fetal bovine serum (FBS; Gibco by Thermofisher Scientific, Waltham, MA USA, catalog n°10270106), 1 mM Sodium Pyruvate (Gibco by Thermofisher Scientific, Waltham, MA USA, catalog n°11360039) and 1×MEM Non-essential amino acids solution (Gibco by Thermofisher Scientific, Waltham, MA USA, catalog n°11140035). SKCO1 (ATCC, Manassas, VA USA, catalog n° HTB-39, RRID:CVCL 0626) were propagated in Minimal essential medium (MEM, Gibco by Thermo Fisher Scientific, Waltham, MA USA, catalog n°21090022) supplemented with 10% heat-inactivated low-endotoxin FBS (Gibco by Thermofisher Scientific, Waltham, MA USA, catalog n°10270106) and 2 mM L-glutamine (Thermo Fisher Scientific, Waltham, MA USA, catalog n° 25030024). C80 cells (ECACC, Salisbury, UK catalog n° 12022904, RRID:CVCL_5249) were maintained in Iscove's modified Dulbecco's medium (IMDM, GlutaMAX supplement; Gibco by Thermo Fisher Scientific, Waltham, MA USA, catalog n° 31980022), supplemented with 10% heat-inactivated low-endotoxin FBS (Gibco by Thermofisher Scientific, Waltham, MA USA, catalog n°10270106). SHSY-5Y cells (ATCC, Manassas, VA USA, catalog n° CRL-2266, RRID:CVCL_0019) were maintained in Dulbecco's Modified Eagle Medium Nutrient Mixture F-12 (DMEM-F12, Gibco by Thermo Fisher Scientific, Waltham, MA USA, catalog n° 31330038) supplemented with 10% heat-inactivated low endotoxin FBS (Gibco by Thermofisher Scientific, Waltham, MA USA, catalog n°10270106). BTECs (Grange et al., 2006) were maintained in EndoGRO-MV-VEGF Complete Culture Media Kit (Merck Millipore, Burlington, MA USA, catalog n° SCME003). LL/2 (LLC1) cells (ATCC, Manassas, VA USA, catalog n° CRL-1642, RRID:CVCL_4358) were cultured in Dulbecco's modified Eagle's medium (DMEM, high glucose, GlutaMAX supplement; Gibco by Thermofisher Scientific, Waltham, MA USA, catalog n° 61965059) supplemented with 10% heat-inactivated low-endotoxin fetal bovine serum (FBS; Gibco by Thermofisher Scientific, Waltham, MA USA, catalog n°10270106).

All cell media were ordinarily supplemented with antibiotics (100 U/ml penicillin and 100 μg/ml streptomycin; Gibco by Thermo Fisher Scientific, Waltham, MA USA, catalog n°15140122). Cells were maintained in a 37° C. and 5% CO₂ air incubator and routinely screened for absence of mycoplasma contamination.

### Animal Models

NOD SCID gamma (NSG) mice were from The Jackson Laboratory (Bar Harbor, ME USA; NOD.Cg-Prkdc^{scid} Il2rg^{tm1Wjl}/SzJ, catalog n°005557, RRID: IMSR_JAX:005557). 8-week-old males were used for the experiments.

Tamoxifen-inducible endothelial specific Flvcr1a-null mice (Flvcr1a^{fl/fl};Cdh5-Cre^{ERT2} mice, named Flvcr1a-KO in the text and figures) were generated in laboratory. Briefly, previously generated Flvcr1a^{fl/fl} mice (Vinchi et al., 2014) were crossed with Cdh5-Cre^{ERT2} mice (Tg(Cdh5-cre/ERT2)1Rha, MGI ID: 3848982, RRID:MGI:3848984), kindly provided by Ralf H. Adams (Sörensen et al., 2009), on a C57BL/6 background. Mice were genotyped by polymerase chain reaction (PCR) analyses on DNA from tail biopsies. To detect the Cdh5-Cre allele, primers Cre-Fw (5′-ACACCTGCTACCATATCATCCTAC-3′-SEQ ID No.: 4) and Cre-Rev (5′-CATCGACCGGTAATGCAG-3′-SEQ ID No.: 5) were used. To analyze the LoxP sites on Flvcr1 gene, primers ILox-Fw (5′-TCTAAGGCCCAGTAGGACCC-3′-SEQ ID No.: 6) and ILox-Rev (5′-GAAAGCATTTCCGTCCGCCC-3′-SEQ ID No.: 7) were used, given a 280-bp band for the floxed allele and a 242-bp band for the wild-type allele. To inactivate Flvcr1a selectively in endothelial cells, 6-week-old Flvcr1a^{fl/fl};Cdh5-Cre^{ERT2} males were treated intraperitoneally with 1 mg/day tamoxifen (Sigma Aldrich St. Louis, MO USA, catalog n°T5648) for 3 consecutive days, followed by 3 additional days after a 4-days treatment free interval. To detect the Flvcr1a-null allele resulting from Cdh5-Cre activity, primers ILox-Fw (5′-TCTAAGGCCCAGTAGGACCC-3′-SEQ ID No.: 8) and IILox-Rev (5′-AGAGGGCAACCTCGGTGTCC-3′-SEQ ID No.: 9) were used, given a 320-bp fragment. Tamoxifen-treated Flvcr1a^{fl/fl} mice were used as control.

All the mice were provided with food and water ad libitum. Experiments were performed on males.

All experiments with animals were approved by the Italian Ministry of Health.

### Analyses on Human Samples Databases

The frequency of tumours showing upregulated FLVCR1 expression with respect to their matched normal tissue was determined using the online tool BioXpress (Wan et al., 2015) (https://hive.biochemistry.gwu.edu/bioxpress). Only those samples that have matched normal tissue expression data were used for this analysis. Numbers of patients overexpressing FLVCR1 in tumor relative to total number of patients examined for that tumor subtype are indicated in the figure. A binomial test was performed to assess the statistical significance of the number of patients overexpressing FLVCR1 in tumor relative to the total number of patients for that tumor subtype, the null hypothesis being equal probability of FLVCR1 being up or down-regulated in cancer for each patient.

### Gene Silencing and Overexpression

FLVCR1a silencing was performed as described in (Destefanis et al., 2019). Briefly, different shRNAs for the human FLVCR1 transcripts were tested. The inventors selected one shRNA specifically down-regulating FLVCR1a, without targeting the FLVCR1b isoform (TRC Lentiviral pLKO.1 Human FLVCR1 shRNA set RHS4533-EG28982, clone TRCN0000059599-SEQ ID No.: 2 shown in FIG. 12; Dharmacon, Lafayette, CO, USA).

To specifically down-regulate ALAS1, two different shRNAs targeting the human ALAS1 transcript were used (TRC Lentiviral pLKO.1 Human ALAS1 shRNA set RHS4533-EG211, clone TRCN0000045740-SEQ ID No.: 10, named shRNA ALAS1 in FIG. 13, and TRCN0000045738-SEQ ID No.: 12, named shRNA ALAS1₂ in FIG. 13; Horizon Discovery, Cambridge, UK).

For control cells, a pLKO.1 lentiviral vector expressing a scramble shRNA (TRC Lentiviral pLKO.1 Non-targeting Control shRNA, catalog n° RHS6848; Dharmacon, Lafayette, CO, USA) was used.

Gene overexpression was performed as described in (Bertino et al., 2019). Briefly, the FLVCR1a cDNA present in pCMV-SPORT6 vector (MGC human FLVCR1 sequence, clone 4417876, Horizon Discovery, Cambridge, UK, catalog n° MHS6278-202757940) and the Myc-tag sequence present in pcDNA3.1(−)/myc-His B (Thermofisher Scientific, Waltham, MA USA, catalog n° V85520) were cloned in frame (SEQ ID No.: 14) in the pLVX-puro vector, a lentiviral expression vector with constitutively active human cytomegalovirus immediate early promoter and the puromycin resistance gene (Clontech Laboratories Inc. A Takara Bio Company, Mountain View, CA, USA, catalog number 632164), in order to obtain the pLVX-puro FLVCR1a vector. For control cells, a pLVX-puro empty vector (Clontech Laboratories Inc. A Takara Bio Company, Mountain View, CA, USA, catalog number 632164) was used [FIG. 11].

Following lentiviral transduction, cells were maintained in selective medium containing 0.002 mg/ml puromycin (Puromycin dihydrochloride from Streptomyces alboniger, Sigma-Aldrich, St. Louis, MO USA, catalog n° P8833).

### Tumor-Associated Endothelial Cells Isolation

Tumor-associated endothelial cells (TECs) were isolated from Lewis lung carcinoma xenografted subcutaneous tumors in tamoxifen-inducible endothelial specific Flvcr1a-null mice (Flvcr1a^{fl/fl};Cdh5-Cre^{ERT2} mice, named Flvcr1a-KO in the text and figures) or control (Flvcr1a^{fl/fl}) mice. Briefly, tumors were dissected and minced into 1-2 mm fragments with a scalpel. Tissue pieces were incubated at 37° C. for 60 minutes in 10 mL of pre-warmed Dulbecco's Phosphate Buffered Saline (DPBS) with Calcium and Magnesium (Lonza Pharma & Biotech, Basel, CH, catalog n° BE17-513F) and 2 mg/ml Collagenase (Collagenase from Clostridium histolyticum, Type I, Sigma Aldrich St. Louis, MO USA, catalog n°C0130), with regular shaking until a single cell suspension was obtained. During this incubation, the cells were mechanically dissociated at 10 minutes intervals by pipetting. To stop the collagenase activity, DMEM (Gibco by Thermofisher Scientific, Waltham, MA USA, catalog n° 61965059) containing 10% FBS (Gibco by Thermofisher Scientific, Waltham, MA USA, catalog n°10270106) was added to the cell suspension, gently pelleted, and rinsed with PBS. The cells in PBS were then filtered through a 40 μm Cell Strainer (Corning Life Sciences, Corning, NY USA, catalog n°352340). Single-cell suspension was centrifuged at 300×g for 10 minutes and tumor-associated endothelial cells were isolated through MACS Technology by using nano-sized MicroBeads, following the manufacturer instructions. Particularly, a negative selection was performed using CD45 MicroBeads (Miltenyi Biotec, Bergisch Gladbach, DE, catalog n°130-052-301). CD45-negative cell fraction was then pelleted and incubated with CD31 MicroBeads (Miltenyi Biotec, Bergisch Gladbach, DE, catalog n°130-097-418) to obtain endothelial cells.

### Xenograft Tumor Model

For the SKCO1 xenograft model, 8-week-old NOD SCID gamma (NSG) male mice (The Jackson Laboratory, Bar Harbor, ME USA; NOD.Cg-Prkdc^{scid} Il2rg^{tm1Wjl}/SzJ, catalog n° 005557, RRID: IMSR_JAX:005557) were housed in a temperature-controlled, pathogen-free environment and used for experimentation. The mice were randomly divided into two groups (n=6 per group). 5*10⁶ SKCO1 cells, stably expressing a shRNA to FLVCR1a or to a scramble control sequence, were suspended in a solution 50% v/v of PBS and matrigel (Corning Matrigel Basement Membrane Matrix, Corning Life Sciences, Corning, NY USA, catalog n° 354234) and then subcutaneously injected into the right flank of mice. After 11 weeks, mice were sacrificed and tumors were harvested. Tumor length (L) and width (W) were measured and tumor volume (mm³) was calculated using the following formula: (L×W²)/2.

For the Lewis lung carcinoma xenograft model, 5×10⁵ LL/2 (LLC1) (ATCC: CRL-1642) murine cells suspended in 100 μl PBS were injected subcutaneously into the flanks of immunocompetent syngeneic (C57BL/6) tamoxifen-inducible endothelial specific Flvcr1a-null mice (Flvcr1a^{fl/fl};Cdh5-Cre^{ERT2} mice, named Flvcr1a-KO in the text and figures) or control (Flvcr1a^{fl/fl}) mice. Both control and inducible knockout mice were treated intraperitoneally with tamoxifen (Sigma Aldrich St. Louis, MO USA, catalog n°T5648; 1 mg/day for 3 consecutive days and 3 additional days after a 4-days treatment free interval) one week before cancer cell injection.

All experimental procedures were approved by the Italian Ministry of Health.

### Hematoxylin and Eosin Staining

Following harvesting, the tissue was washed with 0.1M phosphate-buffered saline (PBS). After overnight fixation in 4% formaldehyde at 4° C., the tissue was decalcified in 0.25M EDTA pH=7.7 for several days and then embedded in paraffin. 5 μm-thick paraffin sections were stained with hematoxylin and eosin (H&E) following standard procedures.

### Cell Viability Assay

To assess cell viability, the CellTiter-Fluor Cell Viability Assay (Promega, Madison, WI USA, catalog n°G6080) was used. The assay is based on measurement of a conserved and constitutive protease activity within live cells and therefore serves as a biomarker of cell viability unrelated to mitochondrial function. The detection of cell viability at different consecutive time points was regarded as a readout of cell proliferation.

### Crystal Violet Staining

For crystal violet staining, 1*10⁴ Caco2 cells were seeded in 6-well plates. Staining was performed at different time points for each cell group in triplicate. Before staining, cells were washed in 0.1M PBS, fixed in 4% paraformaldehyde for 10 minutes. After PFA removal, a 0.1% crystal violet (Sigma-Aldrich, St. Louis, MO USA) solution was applied for 10 minutes under room temperature. The plates were rinsed with water until non color came off and then dried overnight at room temperature. Stained area was assessed by analyses of four representative photos per day for each triplicate using Fiji open source software (Schindelin et al., 2012) (http://fiji.sc/ RRID:SCR_002285).

### Cell Counting

For cell counting, 1*10⁴ BTECs were seeded in 12-well plates and counted at 24, 48 and 72 hours after plating, as a readout of cell proliferation. Three independent experiments were performed.

### Apoptosis Analysis

For cell apoptosis analyses, cells were synchronized in appropriate medium containing 0.1% FBS. The day after, serum was re-introduced. 48 h hours after serum supplement, 5*10⁵ cells were collected, washed in PBS, resuspended in 10 mM Hepes, 140 mM NaCl, 2.5 mM CaCl2 buffer, and labeled with FITC Annexin 5 (Biolegend, San Diego, CA USA, catalog n°640906) for 20 minutes. Then, 2 μl of propidium iodide (1 mg/ml) (propidium iodide solution 1 mg/ml in water, Sigma-Aldrich, St. Louis, MO USA, catalog n° P4864) was added.

Stained cells were analyzed using a FACSCanto II cytofluorimeter and processed with BD FACSDIVA Software v8.1 (BD Biosciences, Milan, IT; http://www.bdbiosciences.com/instruments/software/facsdiva/index.jsp/ RRID:SCR_001456), acquiring at least 10,000 events per sample or 25,000 cells per sample for cell cycle experiments.

### Hemin, 5-ALA and SA Cell Treatment

Hemin (Hemin Ferriprotoporphyrin IX chloride, Frontier Scientific, Logan, UT USA, catalog n° H651-9) was freshly prepared by dissolution in cell culture-grade Dimethyl sulfoxide (DMSO, Sigma Aldrich, St. Louis, MO USA, catalog n°276855) at a concentration of 4 mM and then diluted in tissue culture medium at a concentration of 25 μM.

5-ALA (5-Aminolevulinic acid hydrochloride, Sigma Aldrich, St. Louis, MO USA, catalog n° A3785) was freshly prepared by dissolution in tissue culture medium at a concentration of 5 mM.

SA (Succinyl-acetone, 4-6 Dioxoheptanoic acid, Sigma Aldrich St. Louis, MO USA, catalog n°D1415) was freshly prepared by dissolution in tissue culture medium at a concentration of 0.5 mM.

All solutions were kept in the dark.

### RNA Extraction and Quantitative Real-Time PCR Analysis

RNA extraction and quantitative real-time PCR analyses were performed as described previously (Destefanis et al., 2019). Briefly, total RNA was extracted using Purelink RNA mini kit (Thermofisher Scientific, Waltham, MA USA, catalog n° 12183018A). Between 500 and 1000 ng of total RNA were transcribed into complementary DNA (cDNA) by High-Capacity cDNA Reverse Transcription Kit (Thermofisher Scientific, Waltham, MA USA, catalog n° 4368813). Quantitative real-time PCR (qRT-PCR) was performed using the Universal Probe Library system (Roche, Basel, CH). Primers and probes were designed using the ProbeFinder software (Roche, Basel, CH, https://lifescience.roche.com/en_it/articles/Universal-ProbeLibrary-System-Assay-Design.html/; RRID:SCR_014490). For FLVCR1a, specific primers and the probe were designed using Primer Express Software v3.0 (Thermofisher Scientific, Waltham, MA USA, https://www.thermofisher.com/order/catalog/product/4363991/ RRID:SCR_014326). qRT-PCR were performed on a 7300 or 7900 Real Time PCR System (Thermofisher Scientific, Waltham, MA USA). Transcript abundance, normalized to 18s mRNA expression (for mouse tissues, mouse TECs and for BTECs) or to beta-actin mRNA expression (for cells, except TECs and BTECs), is expressed as a fold increase over a calibrator sample.

### Western Blot Analysis

To assess FLVCR1a and ALAS1 expression, cells were lysed by rotation for 30 minutes at 4° C. in RIPA buffer (150 mM NaCl, 50 mM Tris-HCl pH 7.5, 1% Triton X-100, 0.5% Sodium deoxycholate, 0.1% SDS, 1 mM EDTA). The buffer was freshly supplemented with 1 mM phosphatase inhibitor cocktail (Sigma Aldrich, St. Louis, MO USA, catalog n° P0044), 1 mM PMSF (Sigma Aldrich, St. Louis, MO USA, catalog n° 93482-50ML-F) and protease inhibitor cocktail (Roche, Basel, CH, catalog n° 04693116001). The cell lysate was clarified by centrifugation for 10 minutes at 4° C. Protein concentration in the supernatant was assessed by Bradford assay. For FLVCR1a detection, to remove protein glycosylation, 10 μg of protein extracts were incubated 10 minutes at 37° C. with 1 μL of PNGase-F from Elizabethkingia meningoseptica (Sigma Aldrich, St. Louis, MO USA, catalog n° P-7367). Before loading on 4-15% mini-PROTEAN TGX precast gel (Bio-Rad, Hercules, CA USA, catalog n°4568084), samples were incubated 5 minutes at 37° C. (for FLVCR1a detection) or 5 minutes at 95° C. (for ALAS1 detection) in 4× laemmli buffer freshly supplemented with 8%2-mercaptoethanol. The primary antibodies and dilutions are as follows: mouse monoclonal anti-FLVCR1 (C-4) (Santa Cruz Biotechnology, Dallas, TX USA, catalog n° sc-390100; 1:500), mouse monoclonal anti-ALAS-H (F5) (Santa Cruz Biotechnology, Dallas, TX USA, catalog n° sc-137093; RRID: AB_2225634; 1:1000) and mouse monoclonal anti-Vinculin (Sigma Aldrich, St. Louis, MO USA, catalog n° SAB4200080; RRID: AB_10604160, 1:8000). The revelation was assessed using the ChemiDoc Imaging System (Bio-Rad, Hercules, CA USA).

### Measurement of Heme Concentration

Intracellular heme concentration was measured using a fluorescence assay, as previously reported (Sinclair et al., 2001). Briefly, cells untreated or treated for different times with 5-ALA or SA were collected and 2M oxalic acid was added to them. Samples were heated at 95° C. for 30 minutes leading to iron removal from heme. Fluorescence (wavelength: excitation 405 nm-emission 660-720 nm) of the resultant protoporphyrin was assessed on a Glomax Multi Detection System (Promega Corporation, Madison WI, USA).

The endogenous protoporphyrin content (measured in parallel unheated samples in oxalic acid) was subtracted. Data were normalized to total protein concentration in each sample. Results were expressed as relative fluorescence intensity or, alternatively, as pmol of heme/mg total protein.

### ¹³C-Isotope Labelling Experiments

FLVCR1a-silenced and scramble shRNA-expressing cells were plated in six-well plates in 25 mM glucose, 4 mM glutamine and 1 mM pyruvate-containing DMEM (DMEM, high glucose, pyruvate; Gibco by Thermofisher Scientific, Waltham, MA USA, catalog n°11995-065) supplemented with 10% FBS at 3×10⁵ cells per well. After 24 hours the medium was replaced with 10% FBS- and 1 mM pyruvate-supplemented DMEM containing 4 mM unlabelled glutamine (DMEM, no glucose; Gibco by Thermofisher Scientific, Waltham, MA USA, catalog n°11966-025) and 25 mM ¹³C₆-glucose (D-Glucose-¹³C₆, Santa Cruz Biotechnology, Dallas, TX USA, catalog n° sc-239643B) for ¹³C₆-glucose-tracing experiments; alternatively, the medium was replaced with 10% FBS-supplemented DMEM containing 25 mM unlabelled glucose (DMEM, high glucose, pyruvate, no glutamine, Gibco by Thermofisher Scientific, Waltham, MA USA, catalog n°21969-035) and 4 mM ¹³C₅-glutamine (L-Glutamine-¹³C₅, Sigma Aldrich, St. Louis, MO USA, catalog n°605166), for ¹³C₅-glutamine tracing experiments. The labelled medium was maintained for time indicated in each figure caption. At the end of the incubations, monolayers were rapidly washed three times with ice-cold PBS and extracted with 600 μl of ice-cold extraction solution, composed of methanol, acetonitrile and Milli-Q water (5:3:2), for endo-metabolite determination. Cell extracts were centrifuged at 16,000×g for 20 minutes at 4° C. and the supernatants were analysed by liquid chromatography-mass spectrometry (LC-MS). An Exactive Orbitrap mass spectrometer (Thermofisher Scientific, Waltham, MA USA) was used together with a Thermo Scientific Accela HPLC system. The HPLC setup consisted of a ZIC-PHILIC column (SeQuant, 150 mm×2.1 mm, 5 μm, Merck KGaA, Darmstadt, DE) with a ZIC-PHILIC guard column (SeQuant, 20 mm×2.1 mm, Merck KGaA, Darmstadt, DE) and an initial mobile phase of 20%20 mM ammonium carbonate, pH 9.4 and 80% acetonitrile. Cell extracts (5 μl) were injected and metabolites were separated over a 15-minutes mobile phase gradient, decreasing the acetonitrile content to 20%, at a flow rate of 200 μl min-1 and a column temperature of 45° C. The total analysis time was 23 minutes. All metabolites were detected across a mass range of 75-1,000 m/z using the Exactive mass spectrometer at a resolution of 25,000 (at 200 m/z), with electrospray ionization and polarity switching. Lock masses were used and the mass accuracy obtained for all metabolites was below 5p.p.m. Data were acquired with Thermo Xcalibur software (Thermofisher Scientific, Waltham, MA USA, https://www.thermofisher.com/order/catalog/product/OPTON-30965#/OPTON-30965). The peak areas of different metabolites were determined with Thermo LCQUAN software (Thermofisher Scientific, Waltham, MA USA, https://www.thermofisher.com/order/catalog/product/LCQUAN25?SID=srch-srp-LCQUAN25#/LCQUAN25?SID=srch-srp-LCQUAN25), identified by the exact mass of each singly charged ion and by the known retention time on the HPLC column, obtained by running commercial standards of all metabolites detected. The ¹³C labelling patterns were determined by measuring peak areas for the accurate mass of each isotopologue of several metabolites. Peak areas of each metabolite were normalized to the protein content in each well measured, at the end of the experiment, using the Lowry assay.

### Mitochondria Isolation

According to (Wibom et al., 2002), cells were washed twice in ice-cold 0.1M phosphate-buffered saline (PBS), then lysed in 0.5 mL buffer A (50 mmol/L Tris, 100 mmol/L KCl, 5 mmol/L MgCl₂, 1.8 mmol/L ATP, 1 mmol/L EDTA, pH 7.2), supplemented with protease inhibitor cocktail III [100 mmol/L AEBSF, 80 mmol/L aprotinin, 5 mmol/L bestatin, 1.5 mmol/L E-64, 2 mmol/L leupeptin and 1 mmol/L pepstatin (Merck KGaA, Darmstadt, DE)], 1 mmol/L phenylmethylsulfonyl fluoride (PMSF), 250 mmol/L NaF. Samples were clarified by centrifuging at 650×g for 3 minutes at 4° C., and the supernatant was collected and centrifuged at 13,000×g for 5 minutes at 4° C. The new supernatant was discarded, the pellet containing mitochondria was washed in 0.5 mL buffer A and re-suspended in 0.25 mL buffer B (250 mmol/L sucrose, 15 μmol/L K₂HPO₄, 2 mmol/L MgCl₂, 0.5 mmol/L EDTA, 5% w/v bovine serum albumin). A 100 μL aliquot was sonicated and used for the measurement of protein content and the enzymatic activities of citrate synthase, α-ketoglutarate dehydrogenase, succinate dehydrogenase and malate dehydrogenase. The remaining not-sonicated part was used to measure the electron transport chain (ETC) complexes I-IV activities.

### Citrate Synthase, α-Ketoglutarate Dehydrogenase, Succinate Dehydrogenase and Malate Dehydrogenase Activities

The enzymatic activities of citrate synthase, α-ketoglutarate dehydrogenase, succinate dehydrogenase and malate dehydrogenase were measured on 10 μg mitochondrial proteins using the Citrate Synthase Assay Kit (Sigma Aldrich, St. Louis, MO USA, catalog n° MAK193), Alpha Ketoglutarate (alpha KG) Assay Kit (Abcam, Cambridge, UK, catalog n° ab83431), Malate Dehydrogenase Assay Kit (Sigma Aldrich, St. Louis, MO USA, catalog n° MAK196), Succinate Dehydrogenase Activity Colorimetric Assay Kit (BioVision, Milpitas, CA USA, catalog n° K660), as per manufacturer's instructions. Results were expressed as mU/mg mitochondrial proteins.

### Activity of Mitochondrial ETC Complexes I-IV

The activity of mitochondria respiration complexes was measured according to (Wibom et al., 2002).

### ATP Levels in Mitochondria and Activity of Mitochondrial ATP-Synthase

ATP levels in mitochondria extracts were assessed by the ATP Bioluminescent Assay Kit (Sigma-Aldrich, St. Louis, MO USA, catalog n° FLAA). In this case, ATP was quantified as relative light units (RLU) and converted into nmol ATP/mg mitochondrial proteins, according to the calibration curve previously set.

### Tricarboxylic Acid (TCA) Cycle Flux

The glucose flux through TCA cycle was measured by radiolabeling cells with 2 μCi/ml [6-¹⁴C]-glucose (55 mCi/mmol; PerkinElmer, Waltham, MA, USA, catalog n° NEC045X). Cell suspensions were incubated for 1 hour in a closed experimental system to trap the ¹⁴CO₂ developed from [¹⁴C]-glucose. The reaction was stopped by injecting 0.5 ml of 0.8N HClO₄. The amount of glucose transformed into CO₂ through the TCA cycle was calculated as described (Riganti et al., 2004) and expressed as pmoles CO₂/h/mg cell proteins.

### Wound Healing Assay

To evaluate cell motility, BTECs were grown to confluence in 24-well plates coated with 1% Gelatin from bovine skin (Sigma-Aldrich, St. Louis, MO USA, catalog n° G9391). Then, using a pipette tip, a wound was generated in the middle of the confluent cell monolayer. Floating cells were removed by washing twice with PBS. Cell migration into the wound was monitored using a Nikon Eclipse T-E microscope with a 4× objective. Images were acquired every 2 hours using MetaMorph Microscopy Automation and Image Analysis Software (http://www.moleculardevices.com/Products/Software/Meta-Imaging-Series/MetaMorph.html; RRID:SCR_002368). In the meantime, cells were maintained at 37° C. and 5% CO₂ and did not undergo any significant degree of cell division. Three separate wells were used for each condition and, in each well, at least six fields were analysed for each condition. Cell migration into the wound was assessed using Fiji open source software (Schindelin et al., 2012) (http://fiji.sc/ RRID:SCR_002285), by measuring the distance between the two sides of the wound at each time point. Data were expressed as percentage of cell migration. Three independent experiments were performed.

### Tubulogenesis Assay

To assess in vitro formation of capillary-like structures, 3.5*10⁴ BTECs were seeded in 24-well plates coated with BD Matrigel Matrix Growth Factor Reduced (BD Biosciences, Franklin Lakes, NJ USA, catalog n°356230). Cell organization on Matrigel was monitored using a Nikon Eclipse T-E microscope with a Nikon Plan 10×/0.10 NA objective. Images were acquired every 2 hours using MetaMorph Microscopy Automation and Image Analysis Software (http://www.moleculardevices.com/Products/Software/Meta-Imaging-Series/MetaMorph.html; RRID:SCR_002368). Images obtained 18 hours after the start of the experiment were analysed using Fiji open source software (Schindelin et al., 2012) (http://fiji.sc/ RRID:SCR_002285) in order to assess the number of nodes (intersections formed by at least three detectable cells) for each field. Each experimental condition was performed in duplicate, and at least five fields for each well were analysed. Three independent experiments were performed.

### Quantification and Statistical Analysis

Sample size, mean and statistical details of experiments can be found in the figure legends.

Statistical analyses were conducted in GraphPad Prism v5.0 and v7.0 (GraphPad Software, Inc., La Jolla, CA USA, https://www.graphpad.com/; RRID:SCR_002798), or reported by the computational tools. No statistical method was used to predetermine sample size in studies.

### REFERENCES

BERTINO, F., FIRESTONE, K., BELLACCHIO, E., JACKSON, K. E., ASAMOAH, A., HERSH, J., FIORITO, V., DESTEFANIS, F., GONSER, R., TUCKER, M. E., ALTRUDA, F., TOLOSANO, E. & CHIABRANDO, D. 2019. Heme and sensory neuropathy: insights from novel mutations in the heme exporter feline leukemia virus subgroup C receptor 1. Pain, 160, 2766-2775.
CHEN, C. P., CHEN, K., FENG, Z., WEN, X. & SUN, H. 2019. Synergistic antitumor activity of artesunate and HDAC inhibitors through elevating heme synthesis. Acta Pharm Sin B, 9, 937-951.
CHIABRANDO, D., CASTORI, M., DI ROCCO, M., UNGELENK, M., GIEßELMANN, S., DI CAPUA, M., MADEO, A., GRAMMATICO, P., BARTSCH, S., HÜBNER, C. A., ALTRUDA, F., SILENGO, L., TOLOSANO, E. & KURTH, I. 2016. Mutations in the Heme Exporter FLVCR1 Cause Sensory Neurodegeneration with Loss of Pain Perception. PLOS Genet, 12, e1006461.
DESTEFANIS, F., FIORITO, V., ALTRUDA, F. & TOLOSANO, E. 2019. Investigating the Connection Between Endogenous Heme Accumulation and COX2 Activity in Cancer Cells. Frontiers in Oncology, 9, 162.
FIORITO, V., FORNI, M., SILENGO, L., ALTRUDA, F. & TOLOSANO, E. 2015. Crucial Role of FLVCR1a in the Maintenance of Intestinal Heme Homeostasis. Antioxid Redox Signal, 23, 1410-23.
GRANGE, C., BUSSOLATI, B., BRUNO, S., FONSATO, V., SAPINO, A. & CAMUSSI, G. 2006. Isolation and characterization of human breast tumor-derived endothelial cells. Oncol Rep, 15, 381-6.
KENNEDY, J. C., POTTIER, R. H. & PROSS, D. C. 1990. Photodynamic therapy with endogenous protoporphyrin IX: basic principles and present clinical experience. J Photochem Photobiol B, 6, 143-8.
MALIK, Z. & LUGACI, H. 1987. Destruction of erythroleukemic cells by photoactivation of endogenous porphyrins. Br J Cancer, 56, 589-95.
PENG, C., SONG, Y., CHEN, W., WANG, X., LIU, X., WANG, F., WU, D., MA, S. & GAO, C. 2018. FLVCR1 promotes the proliferation and tumorigenicity of synovial sarcoma through inhibiting apoptosis and autophagy. Int J Oncol.
PENG, Q., MOAN, J., WARLOE, T., NESLAND, J. M. & RIMINGTON, C. 1992. Distribution and photosensitizing efficiency of porphyrins induced by application of exogenous 5-aminolevulinic acid in mice bearing mammary carcinoma. Int J Cancer, 52, 433-43.
PETRILLO, S., CHIABRANDO, D., GENOVA, T., FIORITO, V., INGOGLIA, G., VINCHI, F., MUSSANO, F., CAROSSA, S., SILENGO, L., ALTRUDA. F., MERLO, G. R., MUNARON, L. & TOLOSANO, E. 2018. Heme accumulation in endothelial cells impairs angiogenesis by triggering paraptosis. Cell Death Differ, 25, 573-588.
RIGANTI, C., GAZZANO, E., POLIMENI, M., COSTAMAGNA, C., BOSIA, A. & GHIGO, D. 2004. Diphenyleneiodonium inhibits the cell redox metabolism and induces oxidative stress. J Biol Chem, 279, 47726-31.
RUSSO, V., ROPERTO, F., TAULESCU, M., DE FALCO, F., URRARO, C., CORRADO, F., MUNDAY, J. S., CATOI, C. & ROPERTO, S. 2019. Expression of the feline leukemia virus subgroup C receptors in normal and neoplastic urothelium of the urinary bladder of cattle associated with bovine papillomavirus infection. Vet Microbiol, 229, 147-152.
SCHINDELIN, J., ARGANDA-CARRERAS, I., FRISE, E., KAYNIG, V., LONGAIR, M., PIETZSCH, T., PREIBISCH, S., RUEDEN, C., SAALFELD, S., SCHMID, B., TINEVEZ, J. Y., WHITE, D. J., HARTENSTEIN, V., ELICEIRI, K., TOMANCAK, P. & CARDONA, A. 2012. Fiji: an open-source platform for biological-image analysis. Nat Methods, 9, 676-82.
SHEN, Y., LI, X., ZHAO, B., XUE, Y., WANG, S., CHEN, X., YANG, J., LV. H. & SHANG, P. 2018. Iron metabolism gene expression and prognostic features of hepatocellular carcinoma. J Cell Biochem.
SHI, L., BUCHNER, A., POHLA, H., PONGRATZ, T., RÜHM, A., ZIMMERMANN, W., GEDERAAS, O. A., ZHANG, L., WANG, X., STEPP, H. & SROKA, R. 2019. Methadone enhances the effectiveness of 5-aminolevulinic acid-based photodynamic therapy for squamous cell carcinoma and glioblastoma in vitro. J Biophotonics, 12, e201800468.
SINCLAIR, P. R., GORMAN, N. & JACOBS, J. M. 2001. Measurement of heme concentration. Curr Protoc Toxicol, Chapter 8, Unit 8.3.
SÖRENSEN, I., ADAMS, R. H. & GOSSLER, A. 2009. DLL1-mediated Notch activation regulates endothelial identity in mouse fetal arteries. Blood, 113, 5680-8.
VANDEKEERE, S., DUBOIS, C., KALUCKA, J., SULLIVAN, M. R., GARCÍA-CABALLERO. M., GOVEIA, J., CHEN, R., DIEHL, F. F., BAR-LEV, L., SOUFFREAU, J., PIRCHER, A., KUMAR, S., VINCKIER, S., HIRABAYASHI, Y., FURUYA, S., SCHOONJANS, L., EELEN, G., GHESQUIÈRE, B., KESHET, E., LI. X., VANDER HEIDEN, M. G., DEWERCHIN, M. & CARMELIET, P. 2018. Serine Synthesis via PHGDH Is Essential for Heme Production in Endothelial Cells. Cell Metab, 28, 573-587.e13.
VINCHI, F., INGOGLIA, G., CHIABRANDO, D., MERCURIO, S., TURCO, E., SILENGO, L., ALTRUDA, F. & TOLOSANO, E. 2014. Heme exporter FLVCR1a regulates heme synthesis and degradation and controls activity of cytochromes P450. Gastroenterology, 146, 1325-38.
WAN, Q., DINGERDISSEN, H., FAN, Y., GULZAR, N., PAN, Y., WU, T. J., YAN, C., ZHANG, H. & MAZUMDER, R. 2015. BioXpress: an integrated RNA-seq-derived gene expression database for pan-cancer analysis. Database (Oxford), 2015.
WANG, J., ZHANG, J., SHI, Y., XU, C., ZHANG, C., WONG, Y. K., LEE, Y. M., KRISHNA, S., HE, Y., LIM, T. K., SIM, W., HUA, Z. C., SHEN, H. M. & LIN, Q. 2017. Mechanistic Investigation of the Specific Anticancer Property of Artemisinin and Its Combination with Aminolevulinic Acid for Enhanced Anticolorectal Cancer Activity. ACS Cent Sci, 3, 743-750.
WANG, Z., MA, K., LIU, C., HU, X., QUE, W., ITO, H., TAKAHASHI, K., NAKAJIMA. M., TANAKA, T., REN, K., GUO, W. Z., YI, S. Q. & LI, X. K. 2021. 5-Aminolevulinic acid combined with sodium ferrous citrate (5-ALA/SFC) ameliorated liver injury in a murine acute graft-versus-host disease model by reducing inflammation responses through PGC1-α activation. Drug Discov Ther, 14, 304-312.
WIBOM, R., HAGENFELDT, L. & VON DÖBELN, U. 2002. Measurement of ATP production and respiratory chain enzyme activities in mitochondria isolated from small muscle biopsy samples. Anal Biochem, 311, 139-51.

1. A method of treating a tumor in a subject, comprising administering to the subject in need thereof a blocker of heme synthesis-export axis as active agent, wherein the blocker of heme synthesis-export axis upmodulates oxidative metabolism of tumor cells by upregulating the tricarboxylic acid cycle and the oxidative phosphorylation.

2. The method according to claim 1, wherein the blocker of heme synthesis-export axis exerts antiproliferative and proapoptotic effects on tumor cells.

3. The method according to claim 1, wherein the tumor treatment comprises a reduction or inhibition of tumor proliferation, tumor angiogenesis, and/or tumor pain.

4. The method according to claim 1, wherein the blocker of heme synthesis-export axis is selected from (i) a feedback inhibitor of ALAS1 enzyme, (ii) a down-regulator of FLVCR1a heme exporter and (iii) a down-regulator of ALAS1 enzyme.

5. The method according to claim 4, wherein the feedback inhibitor of ALAS1 enzyme is selected from 5-aminolevulinic acid, derivatives comprising an ester group and/or an acyl group and salts thereof.

6. The method according to claim 4, wherein the down-regulator of FLVCR1a heme exporter is selected from shRNAs complementary to the FLVCR1a mRNA sequence set forth in SEQ ID. No.: 1, wherein the shRNAs comprise an antisense strand of 19-25 continuous bases, preferably 19-22 continuous bases, and a matching sense strand.

7. The method according to claim 4, wherein the down-regulator of FLVCR1a heme exporter is selected from a shRNA comprising an antisense strand as set forth in SEQ ID No.: 2 and a matching sense strand and analogues thereof.

8. The method according to claim 4, wherein the down-regulator of ALAS1 enzyme is selected from shRNAs complementary to the ALAS1 mRNA sequence set forth in SEQ ID. No.: 15, wherein the shRNAs comprise an antisense strand of 19-25 continuous bases, preferably 19-22 continuous bases, and a matching sense strand.

9. The method according to claim 4, wherein the down-regulator of ALAS1 enzyme is selected from a shRNA comprising an antisense strand as set forth in SEQ ID No.: 10 and 12 and a matching sense strand and analogues thereof.

10. The method according to claim 1, wherein the blocker of heme synthesis-export axis is used as a single active agent or in combination with a different anti-tumor agent.

11. The method according to claim 1, wherein the blocker of heme synthesis-export axis is administered by intra-arterial/intracavitary/intraperitoneal/intrapleural/intrathecal infusion, intravascular/intramuscular injection or intra-tumor injection, inhalation, intratracheal/conjunctival/ear/laryngeal/nasal/bladder/urethral instillation, topical/transdermal application, subcutaneous/oral/rectal administration.

12. The method according to claim 4, wherein the feedback inhibitor or the down-regulator of ALAS1 enzyme is administered together with the down-regulator of FLVCR1a heme exporter in a combination therapy.

13. The method according to claim 4, wherein the feedback inhibitor of ALAS1 enzyme is administered together with at least one iron compound.

14. A method of treating a tumor in a subject, comprising administering to the subject in need thereof a pharmaceutical composition comprising as active agent a blocker of heme synthesis-export axis, wherein the blocker of heme synthesis-export axis upmodulates oxidative metabolism of tumor cells by upregulating the tricarboxylic acid (TCA) cycle and the oxidative phosphorylation (OXPHOS).

15. The method according to claim 14, wherein the blocker of heme synthesis-export axis is selected from (i) a feedback inhibitor of ALAS1 enzyme, (ii) a down-regulator of FLVCR1a heme exporter and (iii) a down-regulator of ALAS1 enzyme.


SEQUENCE LISTING (15 sequences)

SEQ ID NO: 1 - DNA, 5919 bp, homo sapiens
agaccttcatctgttcacgcggtagcgcggattgcggttcgcggcgcgcgccaccggggaaggagcggtgggccgaggggttggaggtggggccccaggaggacctcgggctgtgggccgggagagcggagtcggggagtggggcgggggagcgaggtggcgccggggagcctgggatatggcgcggccagacgatgaggagggggcggcggtggcgcccggacacccgctcgcgaaaggatacctcccgttgccgaggggcgcgcccgttgggaaggagagcgtggagctgcagaacgggcccaaagcgggcaccttcccggtgaatggggccccccgggacagcctcgctgccgcctcgggagttctgggcgggcctcagactccactggccccagaagaggagacccaggcccggctgctgcctgcgggcgcgggagctgagaccccgggggccgagagcagcccgctgccccttacggcgctctccccgcggcgcttcgtggtgctcctgatcttcagcctgtactcgctggtcaacgcctttcagtggatccagtacagcatcattagcaacgtcttcgagggcttctacggtgtcaccttgctgcacatcgactggctgtccatggtgtacatgctggcctacgtgcccctcatcttcccggccacctggctgctggacaccagaggcctgcggctcaccgccctgctgggctccggcctcaactgcctgggtgcctggatcaagtgcggcagtgtgcagcagcatctcttctgggtcaccatgttgggccagtgcttgtgctcggtggcccaggtgttcatcctgggcttgccctcccgcatcgcctcagtgtggtttgggcccaaagaggtgtccacagcttgtgccaccgccgtgctgggcaatcagcttggaactgcagttggctttttgctaccaccagttttagtacccaacacacagaatgacacaaatctcctggcttgtaatatcagcaccatgttttatggaacatcagctgttgccacacttttatttattttaacagcaattgccttcaaagaaaaacctcggtatccaccaagtcaggctcaagcagctcttcaagacagtccccctgaagagtactcctataagaaatcaataagaaacctgtttaaaaacattccttttgtccttctgttgatcacttatggtatcatgactggtgccttttattcagtctcaacgttattaaatcaaatgatattgacatattatgagggagaagaagtcaatgctggaaggattgggctaacgctagtagtagctggaatggtgggctctattctttgtggcttatggctggattatactaaaacatacaaacagactactctgatagtttatattttgtcttttattggaatggttatctttactttcacattggaccttagatatattatcatcgtgtttgttactggaggggtgcttggcttcttcatgactggttacctccctttgggttttgaatttgctgttgaaatcacttaccctgaatctgaaggtacttcatctggtcttcttaatgcttctgcacagatatttggaattttgttcacattggctcaaggaaagctcacatcagactatggtcctaaggcagggaacatttttctctgtgtctggatgtttataggcatcatattaacagcattaatcaagtctgatctgcgaagacacaacataaatataggaattacaaatgttgatgttaaagctataccagctgacagtcccacagaccaagaaccaaaaacggttatgttgtccaagcagtcagaatcagcaatttgaagagaaaggcaaagttactgtcctgtagtaattggggacaatgtgatcatccttggagagagatgtgagcaccaaggctgggtttgtatgtggtgggggaataaacacacttacttgaaaattaccatatgaactctaaatgcataattattattttgcttaattgttaaattaagggaaattttcttaaaattcttctgtttacatcatgttaacactactgtttatctaattagtatccggtttttagtctcatattgtatctgaaagtaagcttcttgacgtttactttttaaaagtcgatgtttttcttttttgtagaaaatggaagcttagaatactttttaaagtgataatatggggtgttcagtccccataagatataatagttcatgcagtttatatattaaagtatccagtggaactaaatgtacaatatattcctaattggctgcctttttcactgtgctgaccagctgttcaagccacttcagtttgagtacaacataccaacatgacactactcacccacaaaggacagcattgggatcaggctttcagatgacctctaagatttttcccatttattgtactcttgttacaaagtactttttaacacatgcagtcaatggctataaaaactattctgtgtacagattctacccagactttggtcttagaattatgttctaattaaggagcctggttacaggttcattctgtcttgagttcttttctgtgctgcctttctatcatggataaatgctaacgctgtattttttcactccaacttgagatagagtagttttgtacccattgccttttttttcttttaaactctctttttttttttttcctgatgtgtaactttctagtaagataatttcatcatgtatgttactggctatttcatgatttcatgtatcacatcgtatattttgccttgaagattcctgaaataaggaaatctataataacttcagatagcctacatgtccccatttagtggagacctcttgaatgctatttgaattctgcagtatcatttttatgaccattctcctttgaggaatactatgcccaggtacatgctctatcagtgtgccgggagagtggattcttttcttcactgcagagtcatcacactgttagaatagtctgctcttttacatgctcaggtagggaaaataggaccaaatatatttccacagtgcctaccactgtgtcatgtttacagtgagagtttaaatattgttgatgtcctgactctgtgagctcatagggagtatcttcatagtaatgacatttgatcagccataaaatttacattatgttcatatgcacccaaaaaagctagtcaggtaatgaatacccttgaagtgaatagcaattttgatttaggcagtgtgttaggccatccttgcattgctataaagaaatatttaggctgggcgtggtggctcacgcctgtaatcccagcactttgggaggctgaagcgtgtggatcacctgaggtcaggagttcaagaccagcctggccaacatggtaaaaccccatctctactaaaaaaaaaaaaaaaaaaaaaaaaaaaaaaaaaggccaggcgcagtgctgtggctcatgcctgtaatctcagcactttgggaggccaaggcaggtggatcacaaagtcaggagttcaaaaccagcctggccaagatggtgaaaccccatctctactaaaaatacaaaaattagcgcagtggcaggcgcctgtaatcccagctactcaggaggctgaggcaggagaatcgcttgaatgcgaatggcagaggttgcagtgagcgaaaatcacactactgcactccagcctgggagataaagtgagactgtctcaaataaataaataaataaataaataaataaataaataaataaataaaacctgaggttgagcacgatggctcacacctatagtcccagcactttgggaggctgaggtgggtggatcatttgaggccaggagttgaagaccagtctggccaacatggcaaaaccccatctctactaaaaatacatacacacacacacacacacacaaattagccgggcatggtggcacacgcctgtagtcccagcttcttgggaagctgaggcatgagaattgcctgaacctggaaggcggaggctacagtgagctgagattgcgccactgcactccagcctgggtgacagagcgagactctgtttcaggaaaaaaagaacagaaatacctgagactgggtaatttataaataaaagaggtttaagtggctcacagttctgcaggctgtgtaagcatggcaccagtgtctgctcggcttctggggagccctcagggagctttactcatggtgaaaggctaagtgggagcagagatgtcacatggcgaagcaggagcaagagagcagggaggtgccacacaccttcaaacaaccagattttgtgaaaactcactcattactgcaaggacagcaccaagccatgagggatccacccgcaggacccaaacaccttctattagggccaaccttcaacattggggattacatttcaatgtgagattggagggacaagtatcccaactatatcaggcagaatatatgcattgatatatattgaaattataacattctctataatcaagtgactgtaaattatgtcgaatctgaccctggggaatgtgttatctaaagaatttgactaagaatcaggaactctgtgtccatgtcctggatgtatttctgtatgttcctataactctggacaaatcattaaacctctctgggcagcgttttcctcatccatgaaatgaatgtgttcaatttgatctcaaagatctttccaaaatattaaatgccattaatcatggaatagaccaaattattgtatcacaggaacttgaatccagccagagaaaaattccctggacaattggataaacaaatgtgattgctaccttatggatacaacggacttttacatttctttttagcctttttagtcagttgagggtaactcaagcagagtatgtccaatcattagcttcctttgatatttacattacaggtaaaaacggccaaaaatatattttaaggtctctccccaaatgtttttatttttaaactatcaatgttgtttaaaataatcatgtacttgttgagttcctgaggtttggaacaaattacacataaaatttagaatactttatttctgaaaagcatatacatatatgttatgtttatttttccttgttgattagaaaggtgatggaatatgtgacaatgcaaaatgaattgataatttttctgtattttgagtgaaagttgtctgtaatatgtcaagcaagaatgttataattctacagtaatgtgtgacttcatgacagagctacattctgagaaatttgtcattaggtgatttcatcattgtgtgaacatcatgaagtgtacttacacaaacctaggtggtagagcctactgcacacctgggctagatggcaaagtctgtcgcttctgggctacagacctgtacagcatggtactgtattgaatactgtaggcaactgtaacacaatggtatctgtgtaatctaaacatagaacagataatacattgtgctacaatgtaacaatggctgtggcatcactaggtgataggaatttttcagttccattataatcttataggatctctgtcatatgtggtcaattgttgatcgaaacatgactgtatgtcgtattttcagaaaatggaataggtaatcatcacttgtgtgaattttaatcaaatgacttaggaaagaaactggatgtttcaaaagctgttgcatttattacaaatgtcacaaatacagctcttgccttttgagaatgttggagagatgtctttaaaaaatatgtttgtgtgtaaaaatgtgtctgtatgcaatagctagaaaaatgcctgtgtcttaagtcattactcatgttctaatttttgttctttgtactatttatctgtatgcttgttcttcagtatttcagactcaaaataaatttatttttttatgttaa

SEQ ID NO: 2 - DNA, 21 bp, artificial
  FLVCR1a shRNA - antisense strand
ttgctaatgatgctgtactgg

SEQ ID NO: 3 - DNA, 21 bp, artificial
  FLVCR1a shRNA - sense strand
ccagtacagcatcattagcaa

SEQ ID NO: 4 - DNA, 24 bp, artificial
  primer Cre-Fw
acacctgctaccatatcatcctac

SEQ ID NO: 5 - DNA, 18 bp, artificial
  primer Cre-Rev
catcgaccggtaatgcag

SEQ ID NO: 6 - DNA, 20 bp, artificial
  primer ILox-Fw
tctaaggcccagtaggaccc

SEQ ID NO: 7 - DNA, 20 bp, artificial
  primer ILox-Rev
gaaagcatttccgtccgccc

SEQ ID NO: 8 - DNA, 20 bp, artificial
  primer ILox-Fw
tctaaggcccagtaggaccc

SEQ ID NO: 9 - DNA, 20 bp, artificial
  primer IILox-Rev
agagggcaacctcggtgtcc

SEQ ID NO: 10 - DNA, 21 bp, artificial
  shRNA ALAS1 (1) antisense strand - TRCN0000045740
aagatgagacactctttctgg

SEQ ID NO: 11 - DNA, 21 bp, artificial
  shRNA ALAS1 (1) sense strand - TRCN0000045740
ccagaaagagtgtctcatctt

SEQ ID NO: 12 - DNA, 21 bp, artificial
  shRNA ALAS1 (2) antisense strand - TRCN0000045738
tttcaaagtgtccataactgc

SEQ ID NO: 13 - DNA, 21 bp, artificial
  shRNA ALAS1 (2) sense strand - TRCN0000045738
gcagttatggacactttgaaa

SEQ ID NO: 14 - DNA, 1883 bp, artificial
  FLVCR1aMYC sequence
gaattcccgggatatcgtcgacggcgcgcgccaccggggaaggagcggtgggccgaggggttggaggtggggtcccaggaggacctcgggctgtgggccgggagagcggagtcggggagtggggcgggggagcgaggtggcgccggggagcctgggatatggcgcggccagacgatgaggagggggcggcggtggcgcccggacacccgctcgcgaaaggatacctcccgttgccgaggggcgcgcccgttgggaaggagagcgtggagctgcagaacgggcccaaagcgggcaccttcccggtgaatgggcccccccgggacagcctcgctgccgcctcgggagttctgggcgggcctcagactccactggccccagaagaggagacccaggcccggctgctgcctgcgggcgcgggagctgagaccccgggggccgagagcagcccgctgccccttacggcgctctccccgcggcgcttcgtggtgctcctgatcttcagcctgtactcgctggtcaacgcctttcagtggatccagtacagcatcattagcaacgtcttcgagggcttctacggtgtcaccttgctgcacatcgactggctgtccatggtgtacatgctggcctacgtgcccctcatcttcccggccacctggctgctggacaccagaggcctgcggctcaccgccctgctgggctccggcctcaactgcctgggtgcctggatcaagtgcggcagtgtgcagcagcatctcttctgggtcaccatgttgggccagtgcttgtgctcggtggcccaggtgttcatcctgggcttgccctcccgcatcgcctcagtgtggtttgggcccaaagaggtgtccacagcttgtgccaccgccgtgctgggcaatcagcttggaactgcagttggctttttgctaccaccagttttagtacccaacacacagaatgacacaaatctcctggcttgtaatatcagcaccatgttttatggaacatcagctgttgccacacttttatttattttaacagcaattgccttcaaagaaaaacctcggtatccaccaagtcaggctcaagcagctcttcaagacagtccccctgaagagtactcctataagaaatcaataagaaacctgtttaaaaacattccttttgtccttctgttgatcacttatggtatcatgactggtgccttttattcagtctcaacgttattaaatcaaatgatattgacatattatgagggagaagaagtcaatgctggaaggattgggctaacgctagtagtagctggaatggtgggctctattctttgtggcttatggctggattatactaaaacatacaaacagactactctgatagtttatattttgtcttttattggaatggttatctttactttcacattggaccttagatacattatcatcgtgtttgttactggaggggtgcttggcttcttcatgactggttacctccctttgggttttgaatttgctgttgaaatcacttaccctgaatctgaaggtacttcatctggtcttcttaatgcttctgcacagatatttggaattttgttcacattggctcaaggaaagctcacatcagactatggtcctaaggcagggaacatttttctctgtgtctggatgtttataggcatcatattaacagcattaatcaagtctgatctgcgaagacacaacataaatataggaattacaaatgttgatgttaaagctataccagctgacagtcccacagaccaagaaccaaaaatggttatgttgtccaagcagtcagaatcagcaattccaagctttctagaacaaaaactcatctcagaagaggatctgtgataaaaaatcactagt

SEQ ID NO: 15 - DNA, 2375 bp, homo sapiens
aggctgctcccggacaagggcaacgagcgtttcgtttggacttctcgacttgagtgcccgcctccttcgccgccgcctctgcagtcctcagcgcagttatgcccagttcttcccgctgtggggacacgaccacggaggaatccttgcttcagggactcgggaccctgctggaccccttcctcgggtttaggggatgtggggaccaggagaaagtcaggatccctaagagtcttccctgcctggatggatgagtggcttcttctccacctagattctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtg